SEQUENCE LISTING (12 sequences)

SEQ ID NO: 1 - DNA, 21 bp, Artificial sequence
  Synthetic primer HPRT external 1
gctcgagatgtcatgaaggag

SEQ ID NO: 2 - DNA, 20 bp, Artificial sequence
  Synthetic sequence HPRT internal primer 1
gttctttgctgacctgctgg

SEQ ID NO: 3 - DNA, 20 bp, Artificial sequence
  Synthetic primer HPRT external 2
tccaacacttcgagaggtcc

SEQ ID NO: 4 - DNA, 20 bp, Artificial sequence
  Synthetic primer HPRT internal 2
ggctgtactgcttaaccagg

SEQ ID NO: 5 - DNA, 20 bp, Artificial sequence
  Synthetic primer GATA-3 external 1
gaagctcagtatccgctgac

SEQ ID NO: 6 - DNA, 20 bp, Artificial sequence
  Synthetic primer GATA-3 internal 1
catcgatggtcaaggcaacc

SEQ ID NO: 7 - DNA, 20 bp, Artificial sequence
  Synthetic primer GATA-3 external 2
gggagggtgaagagatgagg

SEQ ID NO: 8 - DNA, 20 bp, Artificial sequence
  Synthetic primer GATA-3 internal 2
gccagagaagaggatgaagc

SEQ ID NO: 9 - DNA, 20 bp, Artificial sequence
  Synthetic primer Tie-2 external 1
gaaacatccctcacctgcat

SEQ ID NO: 10 - DNA, 20 bp, Artificial sequence
  Synthetic primer Tie-2 internal 1
atgaaccagcaccaagatcc

SEQ ID NO: 11 - DNA, 20 bp, Artificial sequence
  Synthetic primer Tie-2 external 2
tgcggcaagtgaacttctaa

SEQ ID NO: 12 - DNA, 20 bp, Artificial sequence
  Synthetic primer Tie-2 internal 2
ccctgtccacggtcatagtt

(19) United States
(12) Patent Application Publication
(10) Pub. No.: US 2010/0055733 A1
(43) Pub. Date: Mar. 4, 2010

(54) MANUFACTURE AND USES OF REACTIVE MICROCONTACT PRINTING OF BIOMOLECULES ON SOFT HYDROGELS

(76) Inventors: Matthias P. Lutolf, St-Sulpice (CH); Helen M. Blau, Menlo Park, CA (US); Regis Doyonnas, Stonington, CT (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/552,400

(22) Filed: Sep. 2, 2009

Related U.S. Application Data
(60) Provisional application No. 61/094,263, filed on Sep. 4, 2008.
(60) Provisional application No. 61/103,990, filed on Oct. 9, 2008.
(60) Provisional application No. 61/116,694, filed on Nov. 21, 2008.

(51) Int. Cl. C12N 5/0789; C12Q 1/02

(52) U.S. Cl. 435/29; 435/396

(57) ABSTRACT

Embodiments of the present disclosure encompass microfabrication methods (“reactive microcontact printing of soft matter”) for hydrated soft polymer materials and surfaces for culture platforms suitable for the culturing of isolated single primary mammalian cells in an environment approximating the natural niches of the cells. Such culture platforms may comprise arrays of microwells, or other microscopically textured features, in which individual features can comprise desired proteins or mixtures of proteins. The microfabrication methods of the disclosure allow spatial control of surface biochemistry and topography at the micrometer scale on these hydrated soft gels. The hydrogels and methods of manufacture and use of the disclosure allow the isolation of a single stem cell and the characterizing of its interaction with cytokines and morphogens, especially with regard to modulation of the proliferative capacity of the stem cell when implanted in a recipient host. The systems for isolating or culturing a eukaryotic cell comprise a hydrogel film comprising a cross-linked polymeric composition having the characteristic of hydrating to form a hydrogel and having a topographical feature or a plurality of topographical features that may have a surface capable of receiving and immobilizing at least one biomolecule species thereon.

### CROSS-REFERENCE TO RELATED APPLICATIONS

This application claims priority to U.S. Provisional Patent Application Ser. No. 61/094,263, entitled “MANUFACTURE AND USES OF REACTIVE MICROCONTACT PRINTING OF BIOMOLECULES ON SOFT HYDROGELS” filed on Sep. 4, 2008; U.S. Provisional Patent Application Ser. No. 61/103,990, entitled “MANUFACTURE AND USES OF REACTIVE MICROCONTACT PRINTING OF BIOMOLECULES ON SOFT HYDROGELS” filed on Oct. 9, 2008; and U.S. Provisional Patent Application Ser. No. 61/116,694, entitled “MANUFACTURE AND USES OF REACTIVE MICROCONTACT PRINTING OF BIOMOLECULES ON SOFT HYDROGELS” filed on Nov. 21, 2008, the entireties of which are hereby incorporated by reference.

### STATEMENT REGARDING FEDERALLY SPONSORED RESEARCH OR DEVELOPMENT

This invention was made with government support under NIH Grant Nos. AG009521, AG020961 and AG024987 awarded by the U.S. National Institutes of Health of the United States government. The government has certain rights in the invention.

### TECHNICAL FIELD

The present disclosure is generally related to methods of microcontact printing soft hydrogels and uses thereof in the isolation and culture of stem cells.

### SEQUENCE LISTING

The present disclosure includes a sequence listing incorporated herein by reference in its entirety.

### BACKGROUND

Rare and fragile primary cells inevitably change their fate and quickly lose their characteristic functions when placed in conventional in vitro tissue culture environments for extended periods of time. Standard in vitro systems such as conventional plastic dish culture systems poorly replicate physiological cell microenvironments with regards to their biochemical and physical properties, or are poorly defined and their characteristics difficult to adapt to particular cells of interest, e.g. biologically derived biopolymer gels that lack tissue and cell specificity. Experimenters also would prefer to expose individual cells, or a controlled number of cells, in in vitro culture settings to well-defined and tunable protein signaling microenvironments to test their effect on “extrinsic” cell regulation.

Current in vitro systems are ill-suited for the culture of adult stem and progenitor cells and other fragile primary cells due to three main restrictions: 1) Adult stem cells can only be isolated with limited purity, even when the most sophisticated phenotypic marker combination and flow cytometry tools are used. The heterogeneous nature of adult stem cell isolates hinders conventional in vitro population-based analysis. Any data characterizing stem cell behavior may be skewed by rapidly overgrowing progenitors. Unicellular systems, in which daughter cells can be analyzed and followed over time at the single cell level as clones, could circumvent this problem. However, current single cell assays rely on standard plastic well formats and in such situations a single cell is difficult to identify and track microscopically on the relatively large surface of a standard 96-well plate; 2) For fragile primary mammalian cells such as adult stem cells from the blood, brain or muscle, the rigid and hydrophobic plastic surface of standard culture plates often, and independently of the lack of any essential signals in the medium, have an adverse effect on cell growth. In addition, adult stem cells may respond to the elasticity of their substrates by changing their fate; aberrant rigid plastic surfaces may favor the commitment of stem cells into undesired lineages; and 3) Current methods of mammalian cell culture do not permit the experimenter to test the effect of multiple proteins and protein compositions in a physiological environment.

Adult stem cells in vivo reside in so-called “niches” or protective microenvironments that are composed of complex mixtures of signaling proteins. A specific microenvironment, or niche, has been shown to play a critical role in the maintenance of stem cell function particularly in Drosophila germ line and mammalian skin (Spradling et al., Nature 414: 98 (2001); Fuchs et al., Cell 116: 769 (2004); Moore & Lemischka, Science 311: 1880 (2006); Scadden, Nature 441: 1075 (2006)). Many essential signals may be membrane-bound and thus conformationally controlled and immobilized on supportive cells in close physical contact with adult stem cells. These signals direct stem cell behavior by different means, protecting them from differentiation, influencing the cell cycle (e.g., maintaining quiescence) and self-renewal divisions. In the absence of cross-talk with their respective natural niche, as is the case with in vitro culture, adult stem cells rapidly differentiate and lose their multipotentiality.

Hematopoiesis relies on the life-long self-renewal and differentiation capacity of sparse populations of hematopoietic stem cells (HSCs). Although HSC function is exemplary in the intact organism, HSCs tend to quickly specialize and lose their stem cell properties when grown in conventional culture conditions. A number of proteins, including Wnt3a and N-cadherin, have been implicated in the HSC niche (Adams & Scadden, Nature Immunology 7: 333 (2006), yet their roles in orchestrating the delicate balance between self-renewal and differentiation remains a matter of debate (Kiel et al., Cell Stem Cell 1: 204 (2007). In vivo studies using knockout and transgenic mouse models have provided important insights (Calvi et al., Nature 425: 841 (2003); Zhang et al., Nature 425: 836 (2003); Arai et al., Cell 118: 149 (2004); Nilsson et al., Blood 106: 1232 (2005); Sugiyama et al., Immunity 25: 977 (2006); Qian et al., Cell Stem Cell 1: 671 (2007, 2007); Yoshihara et al., Cell Stem Cell 1: 685 (2007)), but can also lead to apparently conflicting results due to the complexity of in vivo cell-cell and protein interactions.

In vitro analyses of FACS-enriched HSCs in bulk cultures have been hindered by unavoidable stem cell heterogeneity. However, the responses of single, or isolated, HSCs to protein cues characteristic of the niche, especially during the first few divisions in culture, would shed light on the function of the cells within their respective niches.

### SUMMARY

Embodiments of the present disclosure encompass microfabrication methods (“reactive microcontact printing of soft matter”) for hydrated soft polymer materials and surfaces for culture platforms suitable for the culturing of isolated single primary mammalian cells in an environment approximating the natural niches of the cells. Such culture platforms may comprise arrays of microwells, or other microscopically textured features, in which individual features can comprise desired proteins or mixtures of proteins. The microfabrication methods of the disclosure allow spatial control of surface biochemistry and topography at the micrometer scale on these hydrated soft gels. The hydrogels and methods of manufacture and use of the disclosure allow the isolation of a single stem cell and the characterizing of its interaction with cytokines and morphogens, especially with regard to modulation of the proliferative capacity of the stem cell when implanted in a recipient host. Although not limited, the hydrogels and methods of use thereof of the disclosure are especially advantageous for isolating and proliferating hematopoietic stem cells.

One aspect of the disclosure, therefore, encompasses systems for isolating or culturing a eukaryotic cell, the system comprising a hydrogel film comprising a cross-linked polymeric composition having the characteristic of hydrating to form a hydrogel and having a topographical feature or a plurality of topographical features, where each topographical feature may have a surface capable of receiving and immobilizing at least one biomolecule species thereon. In embodiments of this aspect of the disclosure, the hydrogel film may be hydrated as a hydrogel.

In embodiments of this aspect of the disclosure, the system may further comprise at least one biomolecule species immobilized to the cross-linked polymeric composition. In these embodiments of the disclosure, the biomolecule species may be selected from the group consisting of: a polypeptide, a peptide, an oligonucleotide, and a small molecule. In embodiments of this aspect of the disclosure, the biomolecule may be selected from the group consisting of: Wnt3a, N-cadherin, thrombopoietin, erythropoietin, granulocyte-macrophage colony stimulating factor, granulocyte colony stimulating factor, macrophage colony stimulating factor, thrombopoietin, stem cell factor, interleukin-1, interleukin-2, interleukin-3, interleukin-6, interleukin-7, interleukin-15, Flt3L, leukemia inhibitory factor, insulin-like growth factor, insulin, and recombinant insulin.

In embodiments of the system of the disclosure, the cross-linked polymeric composition may be selected from the group consisting of: a poly(ethylene glycol), a polyaliphatic polyurethane, a polyether polyurethane, a polyester polyurethane, a polyethylene copolymer, a polyamide, a polyvinyl alcohol, a polypropylene glycol, a polytetramethylene oxide, a polyvinyl pyrrolidone, a polyacrylamide, a poly(hydroxyethyl acrylate), and a poly(hydroxyethyl methacrylate)

In the embodiments of the disclosure, a surface of a topographical feature may comprise a reactive functional group of the cross-linked polymeric composition, where the reactive functional group can be capable of binding to the biomolecule species desired to be immobilized on the surface of the topographical feature. In other embodiments of the system of the disclosure, a topographical feature may have a tether immobilized thereon, wherein the tether may be capable of selectively binding to the biomolecule species desired to be immobilized on the surface of the topographical feature.

Another aspect of the disclosure are microcontact printing methods of preparing a hydrogel, comprising: providing a template comprising a negative topographical feature or a plurality of negative topographical features, wherein each negative topographical feature defines a topographical feature desired to be formed in a hydrogel, and wherein the negative topographical feature or features has on the surface thereof a biomolecule species desired to be transferred to a hydrogel, a tether capable of selectively binding to a biomolecule species, or a combination thereof; delivering to the template a hydrogel polymer precursor composition; polymerizing the hydrogel cross-linked polymeric composition to form a hydrogel film; and removing the hydrogel film from the template, thereby transferring the biomolecule species, the tether, or a combination thereof, to a surface of the topographical feature molded in the hydrogel film.

Another aspect of the disclosure is a method of isolating individual cells from a population of cells, wherein the methods may comprise: providing a hydrogel system disposed in a well of a multi-well tissue culture plate, wherein the hydrogel comprises a hydrated cross-linked polymer having an array of topographical features indented therein, and wherein a surface of each of the topographical features has at least one biomolecule species immobilized thereon; delivering a cell suspension of isolated cells to the well of the multi-well plate, whereby the cells of the suspension descend under gravity into the multiplicity of microwells, and wherein the cell density of the cell suspension is adjusted whereby at least one well of the multiplicity of wells receives a single cell; and incubating the hydrogel under conditions favorable for proliferation of the cells.

Still yet another aspect of the disclosure is a method for determining the proliferative outcome of transplanting a stem cell into a recipient host, comprising; delivering a population of cells to a plurality of microwells indented in a hydrogel, wherein an interior surface of each microwell of the plurality of microwells has a biomolecule species immobilized thereon, and wherein at least some of the microwells of the multiplicity of microwells may receive a single cell from the population of cells; monitoring the proliferation of the isolated single cells by time-lapse photography; correlating the proliferation of the cells to the proliferative outcome of a stem cell transplanted into a recipient host; and identifying those cells in a microwell having the characteristic of regenerating when transplanted into a recipient host. In one embodiment of this aspect of the disclosure, the stem cell is a hematopoietic stem cell.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 shows digital images of FACS outputs showing progressive enrichment for Lin⁺c-kit⁺Sca1⁺ (LKS) cells from freshly isolated murine bone marrow cells. Cells were magnetically depleted for Lineage markers and FACS-sorted for Lin⁺c-kit⁺Sca1⁺ (LKS), then resorted twice for CD150⁺ before directly depositing in hydrogel microwell arrays. LKS cells comprised 1%±0.5% of the magnetically-depleted Lin⁺ fraction, and the CD150⁺ fraction comprised 20%±7% of the Lin⁺ c-kit⁺Sca1⁺ LKS cell population.

FIG. 2A is a graph showing an assessment of long-term reconstitution, and therefore the self-renewal potential, of the LKS and LKS-CD150⁺ populations. 10, 20, 40, 100 or 500 GFP⁺ cells (C57BL/6, Ly5.1) of each population were transplanted into lethally-irradiated wild-type host mice (C57BL/6, Ly5.2) together with 500,000 CD150⁻Sca1 helper cells (C57BL/6, Ly5.1). LKS (black bars) and LKS-CD150⁺ (grey bars).

FIG. 2B is a graph showing the percentages of mice that sustained >0.5% of peripheral blood chimerism up to 24 weeks post-transplant for the transplanted populations shown in FIG. 2A.

FIG. 2C shows digital images of FACS outputs of peripheral blood from each transplanted mouse analyzed for reconstitution over a 6 month period by assessing the proportion of GFP⁺Ly5.1⁺ circulating white blood cells within both lymphoid (B220/CD3) and myeloid (Mac1/Gr1) lineages.

FIG. 3 schematically illustrates reactive thiol- and vinylsulfone end-groups on poly(ethylene glycol) (PEG) precursors reacting under mild conditions to form hydrogel matrices that can be used to form microwell arrays.

FIG. 4 schematically illustrates an overview of a multistep process to fabricate hydrogel microwell arrays. Step 1: a PDMS stamp containing an array of micropillars is cast on a silicon master template; Steps 2 and 3: the PDMS stamp is used as template to crosslink a PEG gel containing the complementary microwell array topography; Step 4: upon swelling and washing, the hydrogel surface is used to trap large numbers of individual HSCs. Typical dimensions of a microwell are indicated on the right.

FIG. 5 is a series of digital images showing that a hydrogel microwell array can be placed on the bottom of a well of a standard well plate (here: a 96-well plate) to culture single HSCs (top right) and track their behavior by time-lapse video microscopy over many days (bottom right).

FIG. 6A schematically illustrates a heterofunctional PEG linker used to covalently attach Protein A to the hydrogel network.

FIG. 6B schematically illustrates an overview of a multistep process to locally immobilize Fc-chimeric proteins to the bottom of hydrogel microwells. Steps 1 and 2: a PDMS stamp containing an array of micropillars is inked at the pillar tips with PEG-modified Protein A. Similar to the protein-free process, this stamp is used as a template for molding a PEG gel that contains the complementary microwell array topography. Steps 3 and 4: simultaneously with Steps 1 and 2, PEG-Protein A is transferred to the surface of the forming gel and covalently grafted to tile polymer network. Step 5: upon swelling and washing, an Fc-chimeric protein is incubated and selectively binds to Protein A. Step 6: hydrogel microwell surfaces selectively modified with regulatory proteins of choice are used to trap and study HSCs at the single cell level.

FIG. 6C is a graph showing that stoichiometrically imbalanced hydrogel networks to generate free functional groups (“chemical handles”) for subsequent protein anchoring via multiple bioconjugation strategies.

FIG. 6D shows digital images demonstrating the spatial control in protein immobilization afforded by a hydrogel microcontact printing process. Immobilized FITC-labeled BSA was anchored on the bottom of individual microwells (right panel) rather than on the entire surface of the microwell array (left panel). 3D confocal micrographs of projection of 84 stacks were acquired at a constant slice thickness of 1.8 μm. The small panels below the 3D projections represent (x,z)-cross-sections through the gels revealing the resulting topography.

FIG. 6E schematically illustrates the immobilization of Fc-chimeric proteins to the bottom inside surfaces of microwells via selective binding to Protein A. Alexa-conjugated Fc-fragments and Fc-N-cadherin was tethered and detected via fluorescent microscopy (middle panels). As negative controls (right panels), microwell arrays are shown that are not tethered with Protein A or treated with isotype control primary antibody.

FIG. 7A illustrates a series of digital images of hydrogel microwells where growth of single cells of both LKS-CD150⁺ and LKS populations was monitored via time-lapse video microscopy. Still images selected at the indicated time points were taken from representative movies of LKS (top panels) and LKS-CD150⁺ (bottom panels) cultures. LKS cells were highly proliferative, while LKS-CD150⁺ displayed slow proliferation kinetics. Circles around the wells (top right) indicate microwells hosting clones that underwent different numbers of divisions. Quantification of the distribution of cells per microwell at the indicated time point, as shown in the histograms below each microwell array digital image, confirmed these visual differences. 50% of all microwells comprised 8 or more cells in the LKS population, while 70% of microwells of LKS⁻CD150⁺ contained only 2 cells (220 microwells per condition were analyzed. n=102 cells per histogram for LKS and n=103 for LKS⁻CD150⁺, respectively).

FIG. 7B shows histograms illustrating the distributions of the times to the first division, and times between first and second divisions of single LKS or LKS-CD150⁺ cells (220 microwells per condition analyzed; n=100 and 153 cells per histogram for LKS cells; n=90 and 40 cells per histogram for LKS-CD150⁺, respectively). Stem cells can be distinguished from progenitors by their slow division kinetics.

FIG. 8A shows histograms illustrating the identification of proteins that significantly influence HSC proliferation kinetics at the clonal level. Quantification of the distribution of cells per microwell after 100 hours in culture demonstrated HSC responsiveness to soluble and immobilized Fc-chimeric protein cues characteristic of the HCS niche (n=70-100 microwells/condition and per experiment were analyzed; averages of three independent experiments with standard deviations are shown).

FIG. 8B is a graph illustrating the identification of soluble and tethered putative niche cues that alter proliferation kinetics, compared to basal medium control, via binning of the number of progeny of single HSCs into four groups at 100 hours.

FIG. 8C is a graph showing an additional analysis performed for Type IV proteins to determine the percentage of microwells that contained 3 cells at 24-hour time intervals over a period of one week, compared to basal conditions. Only novel appearances of 3 cells per microwell were scored at each time point to avoid counting the same data twice. T-test for unequal sample size was used (n=57, 175, 65, and 80 microwells for basal, N-cad, Jag-1, and Shh, respectively, ±SEM with the significance level *p<0.05 and **p<0.01).

FIG. 9A schematically illustrates a bulk transplantation assay to assess stem cell function after culture. 100 freshly isolated GFP⁺ HSCs are cultured for at least 4 days in TPO, Wnt3a, N-cadherin or basal medium only (Basal), and all progeny are transplanted into lethally irradiated CD45-congenic C57B16 recipient mice (1 well per recipient mouse, n=10 animals per condition).

FIG. 9B shows graphs relating peripheral blood chimerism in individual recipients as a function of time. Data points represent individual mice repopulated from 3 separate experiments.

FIG. 9C shows a series of digital images of FACS outputs of representative examples of peripheral blood FACS analyses for all conditions.

FIG. 9D shows a pair of graphs illustrating the degree of peripheral blood chimerism in primary and secondary transplants 24 weeks post-transplant for uncultured HSCs, or the progeny of cultured cells in presence of TPO, Wnt3a, N-cad, or basal medium.

FIG. 10A schematically illustrates the transplantation of micro-manipulated clones that have undergone variable division numbers to discriminate between HSC maintenance in the absence or presence of division and expansion. Individual clones tracked in microwells of the microwell arrays by time-lapse video microscopy were selected based on the number of divisions they underwent (no divisions, one division, or more than 3 divisions), picked by micromanipulator, and transplanted into lethally irradiated recipient mice.

FIG. 10B is a graph showing the extents of peripheral blood chimerism in primary transplants 24 weeks post-transplant for singlet cells, doublets (one cell division), or clones (more than 3 cell divisions). Wnt3a and N-cad, but not TPO, maintained HSCs in a stem cell state in the absence of division (singlets) and induces self-renewal divisions (doublets).

The drawings are described in greater detail in the description and examples below.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. Patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions abbreviations are provided and should be used unless otherwise indicated.

### Abbreviations

PB, peripheral blood; PDMS, polydimethylsiloxane; TPO, thrombopoietin; PEG, polyethylene glycol;

### Definitions

In describing and claiming the disclosed subject matter, the following terminology will be used in accordance with the definitions set forth below.

The term “microcontact printing” as used herein refers to the technique whereby a stamp is produced by casting an elastomer such as, but not limited to, an silicon elastomer (for example, polydimethylsiloxane (PDMS)) in the desired pattern which is then coated with a solution of a biomolecule to be transferred to another polymeric structure. After contacting the “inked” stamp with the substrate surface the bio-molecules self-assemble in the pre-given pattern.

The term “polymeric composition” as used herein refers to a single compound species or a mixture of compound species that may be cross-linked to form a polymer. Such precursor compounds include, but are not limited to, such as poly(ethylene glycol), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate) and the like. The polymer compounds before polymerization may be toxic to, or otherwise inhibit the proliferation of a vertebrate cell, but it will be understood by those in the art that when polymerized, the polymer will be inert with respect to any cell or cell line in contact with the polymer.

The term “hydrogel film” as used herein refers to a polymeric material that can absorb at least 10 percent by weight of water when it is fully hydrated. Generally, a hydrogel film material is obtained by polymerization or copolymerization of at least one hydrophilic monomer in the presence of or in the absence of additional monomers and/or macromers.

The term “hydrogel” as used herein refers to a network of polymer chains that are water-insoluble, sometimes found as a colloidal gel in which water is the dispersion medium. Hydrogels can contain over 99% water and may comprise natural or synthetic polymers, or a combination thereof. Hydrogels also possess a degree of flexibility very similar to natural tissue, due to their significant water content. In microstructuring (micromolding) a liquid precursor solution is placed on top of a topographically microstructured rigid surface, for example a silicon or rubber template. The liquid takes on its complementary shape. A cross-linking reaction then transforms the liquid into a solid gel replicating in reverse form the topographical features of the template.

The term “topographical feature” as used herein refers to a protuberance extending from a surface or an indented form extending into a film, gel or other structure. In particular, the features as encompassed herein may take any form that may provide a support and vessel for the culturing of mammalian cells. The term as it applies to indented forms extending into a hydrogel film or hydrated hydrogel, includes, but is not limited to, a cup having a curved apex, a flat-bottomed well or microwell, a groove having a curved base or a flat-base, and the like. A template for microcontact printing and forming a hydrogel film thereon may have protuberances that are the negative or opposite images of the forms to be manufactured in the film or gel. The topographical features such as a microwell may be a multiplicity of features arranged, for example, as an array. For example, a multiplicity of micropillars will form microwells molded into a hydrogel film formed on the template structure.

The term “well” as used herein refers to a well found in a standard tissue culture plate such as a 48- or 96-well plate.

The term “microwell” as used herein refers to wells formed in a hydrogel film or hydrated hydrogel and having a diameter of from about 1 micron to about 500 microns. The term “array of microwells” as used herein refers to a multiplicity (plurality) of microwells arranged in an ordered or random pattern and in close proximity to one another so that a hydrogel having from 2 to about 500 microwells arranged therein can be placed in a well of a standard well plate.

The term “biomolecule species” as used herein refers to any molecule that may be of biological origin and/or interact with a cell in contact therewith. A biomolecule species of use in the systems of the disclosure may be, but are not to be limited to, a protein, a polypeptide, a peptide, a nucleic acid molecule, a saccharide, a polysaccharide, a cytokine and the like that may b, but is not limited to, increasing or decreasing the proliferation of the cell or cell line, may sustain viability and/or proliferation of the cell or cell line, or may initiate a change in the cell type from a stem cell type, a precursor cell type or a progenitor cell type.

The term “protein” as used herein refers to a large molecule composed of one or more chains of amino acids in a specific order. The order is determined by the base sequence of nucleotides in the gene coding for the protein. Proteins are required for the structure, function, and regulation of the body's cells, tissues, and organs. Each protein has a unique function.

The term “heterodimer” as used herein refers to a molecule comprising two identifiable domains or regions having different functions, amino acid sequences, or other properties. The heterodimers useful in the present disclosure may comprise, for example, but not intended to be limiting, a first domain that includes the Fc region of an immunoglobulin and which has an affinity for Protein A, and a second domain comprising such as a cytokine or morphogen intended to interact with a cell disposed in a topographical feature of the hydrogel of the disclosure. The first and second domains may be contiguous, or connected by a linker molecule, wherein the first domain may be linked to the amino or the carboxyl end of the second fragment.

The terms “linker” and “tether” as used herein refer to any molecular structure including, but not limited to, a peptide, a polypeptide, an organic molecular structure able to attach to the surface of a hydrogel film and/or a hydrated hydrogel and bind to a ligand desired to be attached to the hydrogel, or other molecular means whereby a biomolecule may be attached to the surface of the hydrogel. Specific examples include, but are not intended to be limiting, such as a heterofunctional PEG, Protein A that when bound to the hydrogel at one end will specifically bind to the Fc region of an immunoglobulin such as an antibody, Protein G, an immunoglobulin, streptavidin, neutravidin, biotin, a transglutaminase substrate, a peptide that may have a reactive group able to bind to the epsilon-amino groups of lysine residues exposed on the surface of a protein, a Ni²⁺ held by a chelator bonded to the hydrogel, wherein the metal ion can bind a multi-histidine tag of a polypeptide, and the like.

The term “tether” or “linker” may further refer to a molecular structure that conjugates two domains of a heterodimeric polypeptide. It is contemplated that a linker molecule suitable for use in the heterodimeric compositions of the present disclosure, or to link a biomolecule to the hydrogels of the disclosure can be, but is not limited to, a dicarboxylic acid that further includes at least one available group, such as an amine group, for conjugating to a prosthetic group. However, it is also contemplated that other functional side groups may substitute for the amine group to allow for the linking to selected peptides. Exemplary dicarboxylic acids include, but are not limited to, aspartate, glutamate, and the like, and can have the general formula (HOOC)—(CH₂)ₙ—(CHNH₂⁺)—(CH₂)ₘ—(COOH), where n and m are each independently 0, or an integer from 1 to about 10. It is further considered within the scope of the disclosure for the linker to be a multimer, or a combination, of at least two such dicarboxylic acids. For example, such linker molecules may include, but are not limited to, (aspartate)ₓ, (glutamate)_{y}, or a combination thereof, where adjacent amino acids can be joined by peptide bonds, and the like. The subscripts x and y are each independently 0, or an integer from 1 to about 12.

The term “peptide” as used herein refers to short polymers formed from the linking, in a defined order, of α-amino acids. The link between one amino acid residue and the next is known as an amide bond or a peptide bond. Proteins are polypeptide molecules (or consist of multiple polypeptide subunits). The distinction is that peptides are short and polypeptides/proteins are long. There are several different conventions to determine these. Peptide chains that are short enough to be made synthetically from the constituent amino acids are called peptides, rather than proteins, with one dividing line at about 50 amino acids in length.

Modifications and changes can be made in the structure of the peptides of this disclosure and still result in a molecule having similar characteristics as the peptide (e.g., a conservative amino acid substitution). For example, certain amino acids can be substituted for other amino acids in a sequence without appreciable loss of activity. Because it is the interactive capacity and nature of a peptide that defines that peptide's biological functional activity, certain amino acid sequence substitutions can be made in a peptide sequence and nevertheless obtain a peptide with like properties.

As used herein, the terms “oligonucleotide” and “polynucleotide” generally refer to any polyribonucleotide or polydeoxyribonucleotide that may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The terms “nucleic acid,” “nucleic acid sequence,” or “oligonucleotide” also encompass a polynucleotide as defined above.

The term “Michael-type reaction” as used herein refers to the nucleophilic addition of a carbanion to an alpha, beta unsaturated carbonyl compound.

The term “flow cytometer” as used herein refers to any device that will irradiate a particle suspended in a fluid medium with light at a first wavelength, and is capable of detecting a light at the same or a different wavelength, wherein the detected light indicates the presence of a cell or an indicator thereon. The “flow cytometer” may be coupled to a cell sorter that is capable of isolating the particle or cell from other particles or cells not emitting the second light.

The term “proliferative status” as used herein refers to whether a population of cells including, but not limited to, hematopoietic stem or progenitor cells, or a subpopulation thereof, are dividing and thereby increasing in number, in the quiescent state, or whether the cells are not proliferating, dying or undergoing apoptosis.

The terms “modulating the proliferative status” or “modulating the proliferation” as used herein refers to the ability of a compound to alter the proliferation rate of a population of hematopoietic stem or progenitor cells A compound may be toxic, wherein the proliferation of the cells is slowed or halted, or the proliferation may be enhanced such as, for example, by the addition to the cells of a cytokine or growth factor.

The term “quiescent” as used herein refers to cells that are not actively proliferating by means of the mitotic cell cycle. Quiescent cells (which include cells in which quiescence has been induced as well as those cells which are naturally quiescent, such as certain fully differentiated cells) are generally regarded as not being in any of the four phases G1, S, G2 and M of the cell cycle; they are usually described as being in a G0 state, so as to indicate that they would not normally progress through the cycle. Cultured cells can be induced to enter the quiescent state by various methods including chemical treatments, nutrient deprivation, growth inhibition or manipulation of gene expression, and induced to exit therefrom by contacting the cells with cytokines or growth factors.

The term “eukaryotic cell” as used herein refers to a cell as found in the tissues of an animal other than an enucleated erythrocyte.

The term “cell or population of cells” as used herein refers to an isolated cell or plurality of cells excised from a tissue or grown in vitro by tissue culture techniques. In the alternative, a population of cells may also be a plurality of cells in vivo in a tissue of an animal or human host.

The term “cytokine” as used herein refers to any cytokine or growth factor that can induce the differentiation of a hematopoietic stem cell to a hematopoietic progenitor or precursor cell and/or induce the proliferation thereof, and which may be liked to the surface of a soft hydrogel according to the disclosure. Suitable cytokines for use in the present disclosure include, but are not limited to, erythropoietin, granulocyte-macrophage colony stimulating factor, granulocyte colony stimulating factor, macrophage colony stimulating factor, thrombopoietin, stem cell factor, interleukin-1, interleukin-2, interleukin-3, interleukin-6, interleukin-7, interleukin-15, Flt3L, leukemia inhibitory factor, insulin-like growth factor, and insulin. The term “cytokine” as used herein further refers to any natural cytokine or growth factor as isolated from an animal or human tissue, and any fragment or derivative thereof that retains biological activity of the original parent cytokine. The cytokine or growth factor may further be a recombinant cytokine or a growth factor such as, for example, recombinant insulin. The term “cytokine” as used herein further includes species-specific cytokines that while belonging to a structurally and functionally related group of cytokines, will have biological activity restricted to one animal species or group of taxonomically related species, or have reduced biological effect in other species. The term “cytokine” as used herein further includes “morphogen”, which refers to a substance governing the pattern of tissue development and, in particular, the positions of the various specialized cell types within a tissue. It spreads from a localized source and forms a concentration gradient across a developing tissue. In developmental biology a morphogen is rigorously used to mean a signaling molecule that acts directly on cells (not through serial induction) to produce specific cellular responses dependent on morphogen concentration. Well-known morphogens include, but are not limited to, transforming growth factor beta (TGF-β), Hedgehog/Sonic Hedgehog, Wingless/Wnt, epidermal growth factor (EGF), and fibroblast growth factor (FGF), and the like. Morphogens are defined conceptually, not chemically, so simple chemicals such as retinoic acid may also act as morphogens.

The term “primary cell” refers to cells obtained directly from a human or animal adult or fetal tissue, including blood. The “primary cells” or “cell lines” may also be derived from a solid tumor or tissue that may or may not include a hematopoietic cell population, and can be suspended in a support medium. The primary cells may comprise a primary cell line.

The term “primitive hematopoietic cell” as used herein refers to any stem, progenitor or precursor cell that may proliferate to form a population of hematopoietic cells.

The term “hematopoietic stem cells” as used herein refers to pluripotent stem cells or lymphoid or myeloid (derived from bone marrow) stem cells that, upon exposure to an appropriate cytokine or plurality of cytokines, may either differentiate into a progenitor cell of a lymphoid or myeloid cell lineage or proliferate as a stem cell population without further differentiation having been initiated. “Hematopoietic stem cells” include, but are not limited to, colony-forming cell-blast (CFC-blast), high proliferative potential colony forming cell (HPP-CFC) and colony-forming unit-granulocyte, erythroid, macrophage, megakaryocyte (CFU-GEMM) cells, and the like.

The terms “progenitor” and “progenitor cell” as used herein refer to primitive hematopoietic cells that have differentiated to a developmental stage that, when the cells are further exposed to a cytokine or a group of cytokines, will differentiate further to a hematopoietic cell lineage. “Progenitors” and “progenitor cells” as used herein also include “precursor” cells that are derived from some types of progenitor cells and are the immediate precursor cells of some mature differentiated hematopoietic cells. The terms “progenitor”, and “progenitor cell” as used herein include, but are not limited to, granulocyte-macrophage colony-forming cell (GM-CFC), megakaryocyte colony-forming cell (Mk-CFC), burst-forming unit erythroid (BFU-E), B cell colony-forming cell (B-CFC) and T cell colony-forming cell (T-CFC). Precursor cells” include, but are not limited to, colony-forming unit-erythroid (CFU-E), granulocyte colony forming cell (G-CFC), colony-forming cell-basophil (CFC-Bas), colony-forming cell-eosinophil (CFC-Eo) and macrophage colony-forming cell (M-CFC) cells.

“Polymerase chain reaction” or “PCR” refers to a thermocyclic, polymerase-mediated, DNA amplification reaction. A PCR typically includes template molecules, oligonucleotide primers complementary to each strand of the template molecules, a thermostable DNA polymerase, and deoxyribonucleotides, and involves three distinct processes that are multiply repeated to effect the amplification of the original nucleic acid. The three processes (denaturation, hybridization, and primer extension) are often performed at distinct temperatures, and in distinct temporal steps. In many embodiments, however, the hybridization and primer extension processes can be performed concurrently. The nucleotide sample to be analyzed may be PCR amplification products provided using the rapid cycling techniques described in U.S. Pat. Nos. 6,569,672; 6,569,627; 6,562,298; 6,556,940; 6,569,672; 6,569,627; 6,562,298; 6,556,940; 6,489,112; 6,482,615; 6,472,156; 6,413,766; 6,387,621; 6,300,124; 6,270,723; 6,245,514; 6,232,079; 6,228,634; 6,218,193; 6,210,882; 6,197,520; 6,174,670; 6,132,996; 6,126,899; 6,124,138; 6,074,868; 6,036,923; 5,985,651; 5,958,763; 5,942,432; 5,935,522; 5,897,842; 5,882,918; 5,840,573; 5,795,784; 5,795,547; 5,785,926; 5,783,439; 5,736,106; 5,720,923; 5,720,406; 5,675,700; 5,616,301; 5,576,218 and 5,455,175, the disclosures of which are incorporated by reference in their entireties. Other methods of amplification include, without limitation, NASBR, SDA, 3SR, TSA and rolling circle replication. It is understood that, in any method for producing a polynucleotide containing given modified nucleotides, one or several polymerases or amplification methods may be used. The selection of optimal polymerization conditions depends on the application.

The term “primer” as used herein refers to an oligonucleotide, the sequence of at least a portion of which is complementary to a segment of a template DNA which to be amplified or replicated. Typically primers are used in performing the polymerase chain reaction (PCR). A primer hybridizes with (or “anneals” to) the template DNA and is used by the polymerase enzyme as the starting point for the replication/amplification process. By “complementary” is meant that the nucleotide sequence of a primer is such that the primer can form a stable hydrogen bond complex with the template; i.e., the primer can hybridize or anneal to the template by virtue of the formation of base-pairs over a length of at least ten consecutive base pairs.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

### Discussion

### Reactive Microcontact Printing of Biomolecules on Soft Hydrogels

The embodiments of the disclosure encompass microfabrication technologies (“reactive microcontact printing of soft matter”) for hydrated materials that enable the creation of culture platforms overcoming the inherent problems of conventional methods for the culture of isolated populations of primary stem cells. Such culture platforms comprise arrays of microwells or other microscopically textured features with desired dimensions (usually about 10 to about 100 microns) in which individual features can include any desirable tethered or otherwise immobilized protein or mixture of proteins. The microfabrication technology, described in more detail below, allows spatial control of surface biochemistry and topography at the micrometer scale on these hydrated soft polymer surfaces.

Microcontact printing is a versatile technique to obtain micrometer-scale patterned (i.e., biochemical or chemical modifications of surfaces) using soft polymer stamps such as, but not limited to, a stamp comprised of polydimethylsiloxane (PDMS) rubber. A stamp is soaked in molecular “ink” comprised of a desired compound such as a bioactive protein that can be imprinted on a surface to generate a desired pattern. A wide variety of materials including hydrophobic polymers, glass or metals have been successfully patterned with this method. However, microcontact printing to pattern soft, hydrated, biocompatible materials such as hydrogels has proven difficult. Microcontact printing of soft matter has not been possible because the chemical modifications of hydrogel surfaces are very challenging as most covalent chemical schemes used to modify common polymer surfaces to accept a polypeptide are not compatible with an aqueous environment.

In microstructuring (or micromolding) of hydrogels, a liquid precursor solution is placed on top of a topographically microstructured rigid surface, such as a silicon or a rubber template. By wetting the template, the liquid takes on its complementary shape. A cross-linking reaction takes place transforming the liquid into a gel that can retain the desired positive form of the topographical features when the two surfaces are separated.

It may also be desirable that microstructures be generated in combination with a desired biochemical surface pattern. Cellular responses could then be restricted to a particular area on a surface. For example, with highly migratory cells such as blood-derived cells, a uniformally flat and biochemically patterned surface would not prevent cells from leaving an area of interest. However, by localizing the biochemical modifications to a defined area, such as the base of a microwell, the cells are more readily confined.

The present disclosure, therefore, provides methods for topographically microstructuring (“micromolding”) a hydrogel, and to pattern, or imprint, selected regions of the hydrogels with bioactive ligands (“microprinting”) bonded to the hydrophilic polymer gel surfaces.

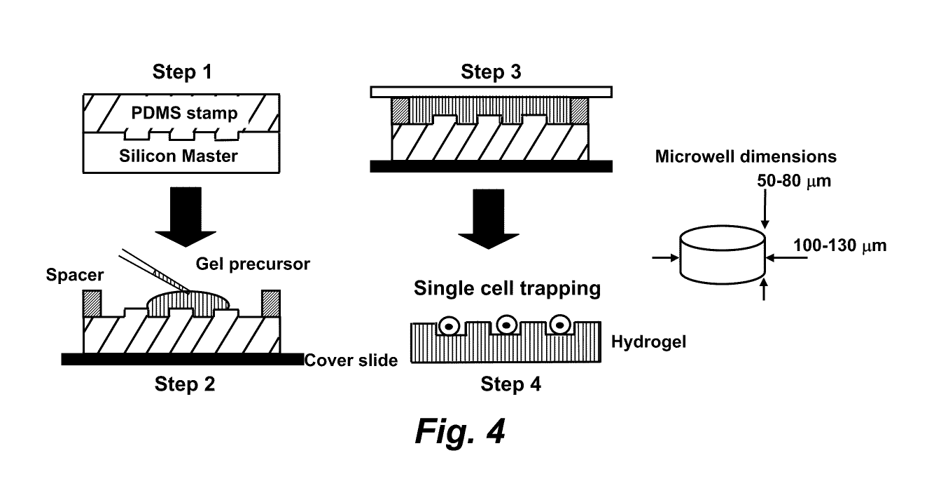

In one embodiment, the first step of the methods of the disclosure, therefore, is the micromolding of hydrogels with a desired topography, such as schematically illustrated in FIG. 4. it is contemplated that a ‘negative’ master mold may be formed from a resilient material such as a silicon polymer. The desired topographical features to be formed in the final hydrogel are first formed in a negative conformation in the master mold or template.

This molding process may be achieved by cross-linking liquid polymer precursors deposited on a pre-structured elastomeric stamp using a polymer such as, but not limited to, polydimethylsiloxane, PDMS and the like, thereby forming a negative replica, or stamp, of the template, but in reverse thereof. The template, which may be a hardened material such as, but not limited to, a silicon wafer, will have protruding from the surface thereof, or indented into, a negative form of a topographical feature or a multiplicity of features desired to be molded into a hydrogel. The template may be coated with such as a silicone-based material to facilitate the removal of a hydrogel film from the template.

A next step in the methods of the disclosure is to mold a hydrogel using the stamp to provide the desired topographical features. For this purpose a polymer solution may be prepared from one or more monomers that together, or in combination, may cross-link to provide a hydrogel polymer with sufficient resilience to withstand removal from the template without damage and to retain the forms of the topographical features molded into the film.

For example, but not intended to be limiting, conjugate addition cross-linking reactions between vinylsulfone end-groups (or other groups containing conjugated unsaturations such as acrylate or maleimide) on branched, multiarm polyethylene glycol (PEG) macromers, and thiol residues on bifunctional PEGs, may be used to form 3-dimensional polymer networks that can absorb large amounts of water.

It is anticipated that the master mold and templates formed therefrom and used in the methods of the disclosure may provide indentations or protuberances into or from the surface of the hydrogel, and that the indentations or protuberances may have any desired configuration (topography) such as, but not limited to, microwells, grooves, irregular shapes, and the like, that may be combined with biochemical patterning. In one example, arrays of microwells with variable dimensions ranging from about 1 micron to about 500 microns, and spacing were fabricated. In another example, grooves with controlled dimension were made. Embodiments of the methods of the disclosure, therefore, may provide a wide variation in the topographical features molded into the hydrogel polymer. Especially useful for the isolation and proliferation of stem cells such as, but not limited to, hematopoietic stem cells, according to the methods of the disclosure, is an array of microwells indented into a final hydrogel.

It is further contemplated that the methods of the disclosure may provide any multi-component protein patterning (i.e., protein “co-localization”). The polymer stamp may be inked with a mixture of proteins that can be readily imprinted onto the forming gel surface. Alternatively, a desired protein, peptide, or other cell effector may be tethered to a hydrogel microwell using a capture group such as Protein A that binds multiple Fc-chimeric proteins with similar affinity. For example, but not intended to be limiting, Protein A may be imprinted to a hydrogel surface such as the base of a microwell. The desired cell effector protein or peptide may be a chimeric polypeptide having an immunoglobulin Fc region that may bind to the Protein A, thereby immobilizing the effector-Fc chimera to the hydrogel surface. In another embodiment, it is contemplated that a cell effector or potential cell effector may be immobilized by first immobilizing and effector-specific antibody to the hydrogel.

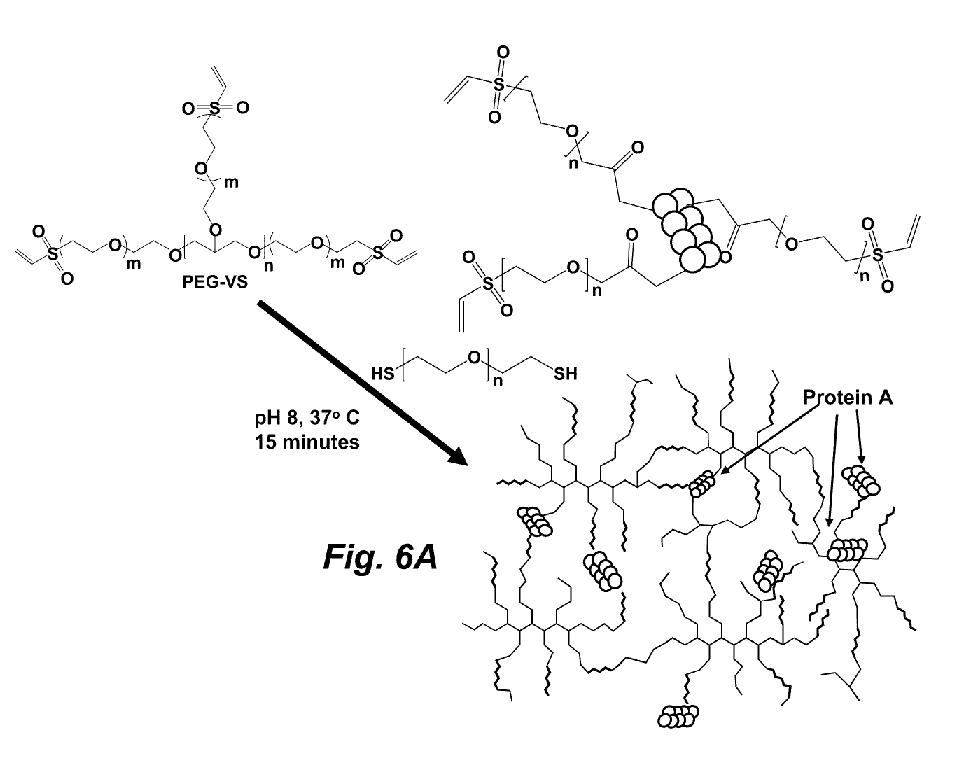

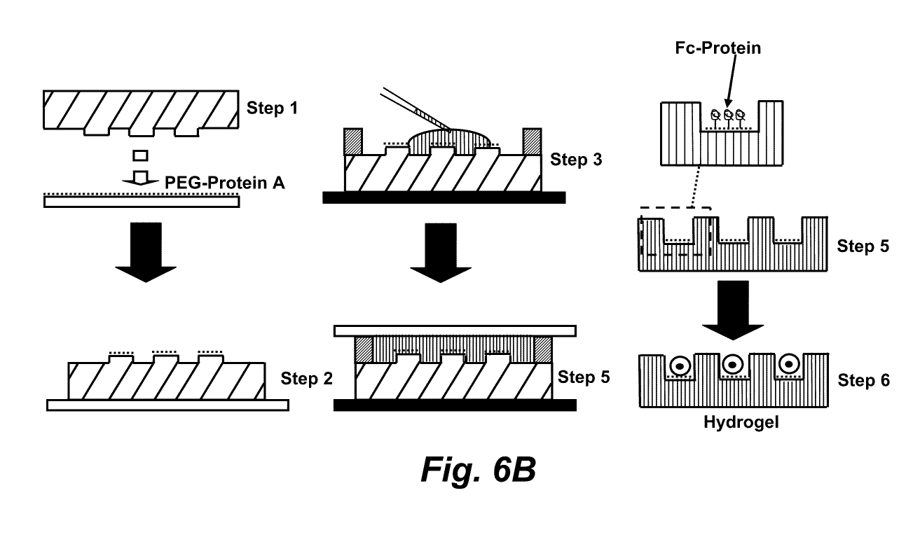

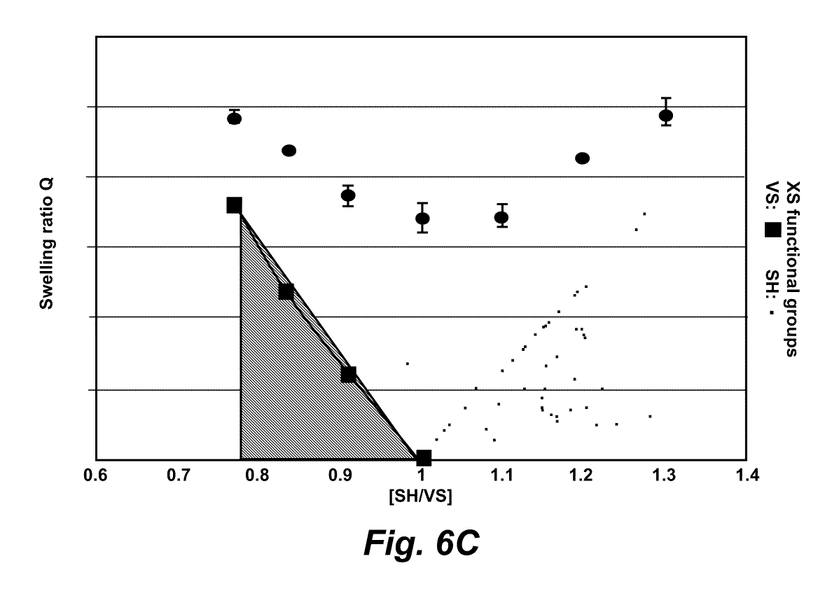

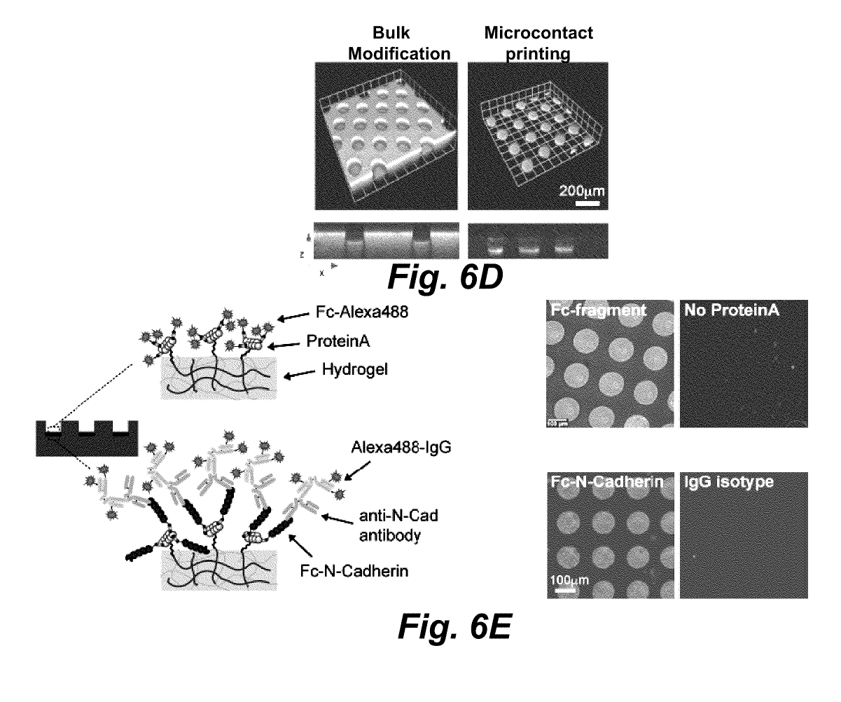

To control both the topography and localized presentation of cell-regulatory proteins on the arrayed microenvironments of the hydrogels of the disclosure, protein immobilization may restricted to selected areas on the surface. This is accomplished by using “reactive microcontact printing”, as schematically shown in FIGS. 6A and 6B. Thus, it is contemplated that, for example, a PDMS template may be first inked with desired proteins, where the proteins can be adsorbed just on the tip of positive (protruding) features (e.g., micropillars, microridges and the like).

Subsequently, polymerization may be conducted on this template, in the course of which the biomolecule species of interest can be transferred from the surfaces of the template to the developing microstructured gel matrix, on which the proteins may become locally surface-tethered. Functional groups on the hydrogel polymer (capture ligands) that provide the “activated surface” are those groups that are not consumed during the cross-linking reaction. The residual capture ligands may result from a stoichiometric imbalance between the reactive functional groups of the polymer (such as vinylsulfone, acrylates, maleimide, thiols, or amines) leaving some unconsumed and therefore available after the polymerization reaction, as shown in FIG. 6C. Alternatively, functional groups of the polymer material available for imprinting are those groups that do not participate in the cross-linking of the polymerization.

The free functional groups can then serve as “capture” ligands for direct or indirect biomolecule tethering, as shown in FIG. 6A. It is contemplated that anchoring of polypeptides may be achieved by direct linkage of the capture ligands of the hydrogel surfaces to a protein (by means of, for example, but not limited to, epsilon-amine groups of lysine residues of the protein). Alternatively, the protein(s) to be bound to the hydrogel surface may be attached indirectly via, for example, a molecule that may act as intermediary tether or linker to bind the target protein by means of non-covalent interactions (e.g., Protein A or Protein G binding to Fc-regions of immunoglobulins, the biotin-streptavidin interaction, Ni⁺-affinity of proteins containing His-tags, and the like).

For example, by deviating from the equal stoichiometry between thiols and vinylsulfones in the hydrogel polymer cross-linking reaction, it is possible to generate an “activated” gel network that also comprises free thiols or vinylsulfones that can function as capture ligands for protein tethering. In one embodiment of the disclosure, for example, one end of a heterofunctional linker may bond to a free thiol or vinylsulfone group of the hydrogel polymer, and have at the opposing end an amine-reactive NHS ester group for binding to a lysine side-chain of a desired protein (FIG. 4). In another embodiment, Protein A may be tethered to hydrogel microwell surfaces to specifically bind engineered, Fc-chimeric proteins of choice, as shown in FIGS. 6A-6E. Biomolecule species tethered via Protein A would then be presented in a conformation that corresponds to their natural state, and hence protein activity would not be perturbed, in contrast to more conventional protein binding to the plastic surfaces of cell culture plates.

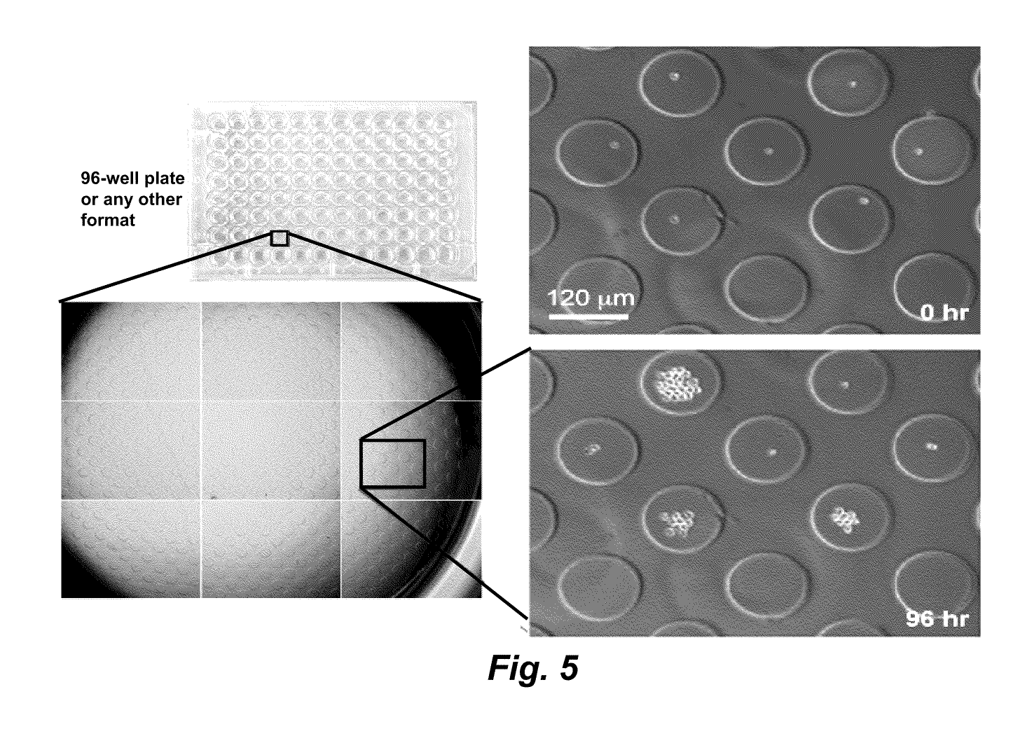

Manufacture of the microprinted hydrogels of the disclosure may be achieved in one step. Since hydrophilic protein and cell-repellent polymers such as PEG are used in the manufacturing methods of the disclosure, “passivation” or blocking of the surface to produce cell-non-adhesive areas is not necessary. Interaction of cells with the hydrogel surface is, therefore, restricted to the protein-patterned regions, as shown in FIG. 5.

It is contemplated that almost any protein can be patterned to the hydrogel surfaces produced by the methods of the disclosure, for example, non-specifically via amine groups, or site-selectively by, for example, protein-protein interactions. Thus, for example, the protein components of physiological stem cell niches including, but not limited to, transmembrane proteins involved in cell-cell adhesion such as cadherins, selectins and CAMS belonging to the Ig superfamily (ICAMs and VCAMs), developmental morphogens including the Notch ligands Jagged and Delta, hedgehog proteins, Wnts, and integrin-binding extracellular matrix proteins such as fibronectin, laminin, integrins, osteopontin and matricellular proteins such as tenascins, may be attached to the hydrogel polymer surfaces directly or indirectly.

Reactive microcontact printing is not limited to poly(ethylene glycol) hydrogel networks, and is useful for a wide variety of other cross-linking chemistries including chemical cross-linking via photopolymerization, or physical cross-linking of gels. Many other synthetic or naturally-derived gelling macromolecules can be used, including, but not limited to, poly(vinyl alcohol), poly(vinylpyrrolidone), polyacrylamides, poly(N-isopropylacrylamide), agarose, gelatin, methylcellulose. The only requirement is free capture groups after polymerization that can be utilized for surface-mediated biomolecule coupling.

Protein concentrations can be controlled. An ELISA-based approach was employed to quantify the protein surface density. It was found that densities as low as in the mid fmol/cm² range can be grafted onto PEG-based gels. Protein surface densities can be adjusted by a change in stoichiometric balance of the network since it is linearly dependent on the number of free capture groups per volume.

This process may also lend itself to the deposition of biomolecules onto the stamp form via commercially available micro- or nano-printers used to fabricate DNA and protein arrays (on flat surfaces). Thereby, more complex gel patterning can be achieved. Using protein printing technologies, the generation of arrayed microenvironments composed of entire protein libraries is possible. Individual microwells could be tethered with distinct protein compositions generating a hydrogel “chip” of artificial cell microenvironments

By using multiple tagged proteins with specific intermolecular binding partners (for example, protein-Fc and biotin-streptavidin, or Protein A-Fc and NPA-His-tag), it is anticipated that complex multi-component protein-patterned microenvironments can be generated that “self-aggregate”. For example, a mixture of two or more proteins, each bearing distinct tags may spontaneously and selectively segregate to previously defined areas on a structured hydrogel upon incubation with a mixture of proteins in solution. A requirement for such directed segregation of polypeptides from a mixture of polypeptides is the generation of spatially defined regions, each having a distinct capture group able to specifically bind to a particular species of the polypeptide in the mixture. Using this approach, the engineering of multi-component signaling systems is possible and which may more closely resemble natural stem cell niches.

It is contemplated that the fabrication of surfaces of soft hydrogels containing gradients of tethered proteins can also be achieved using microfluidics-based gradient generators or other methods. In addition, it is contemplated that the methods of the present disclosure may be adopted for the patterning hydrogels that are not topographically structured. For example, proteins may be printed on planar hydrogels containing free capture groups, similar to the well-known microcontact printing technology on rigid surfaces.

This microfabrication method for soft materials according to the present disclosure is particularly useful in tissue engineering and biotechnology (e.g., cell-based sensors). For example, as described detail below, and in the Examples of the disclosure, arrayed artificial microenvironments may be used in the study of stem cells in response to desirable combinations of tethered proteins. Such hydrogel niche arrays are compatible with conventional cell culture labware in that they may be sized and formed to fit a desired well size (for example, 24-, 48- or 96-well size) and placed at the bottom of a well. For example, 24-well plates are particularly useful when cells or groups of cells need to be recovered with a micromanipulator. A suspension of adult stem cells or progenitor cells can then be sedimented by gravity, stochastically distributing the cells into the microwells. Depending upon the seeding density, either single cells or cell clusters will descend into individual wells and then can be studied in response to desired biomolecules on this platform.

Conventional techniques can be utilized to assay cell function. Due to the transparency of the hydrogel, cells can be studied by live time-lapse microscopy, using bright field or fluorescence. Live cells can be removed from desired wells by micromanipulation for subsequent experiments. They can be fixed and immunostained after cell culture for retrospective phenotypic analyses. Platforms can be generated that are well suited to look at rare events of single cells such as asymmetric stem cell division. On the other hand, platforms that are engineered for high-throughput drug screening purposes on a chip can be fabricated.

Application of Micro-Printed Soft Hydrogels to the Culture of Hematopoietic Stem Cells, and Characterization of their Protein Factor Interactions

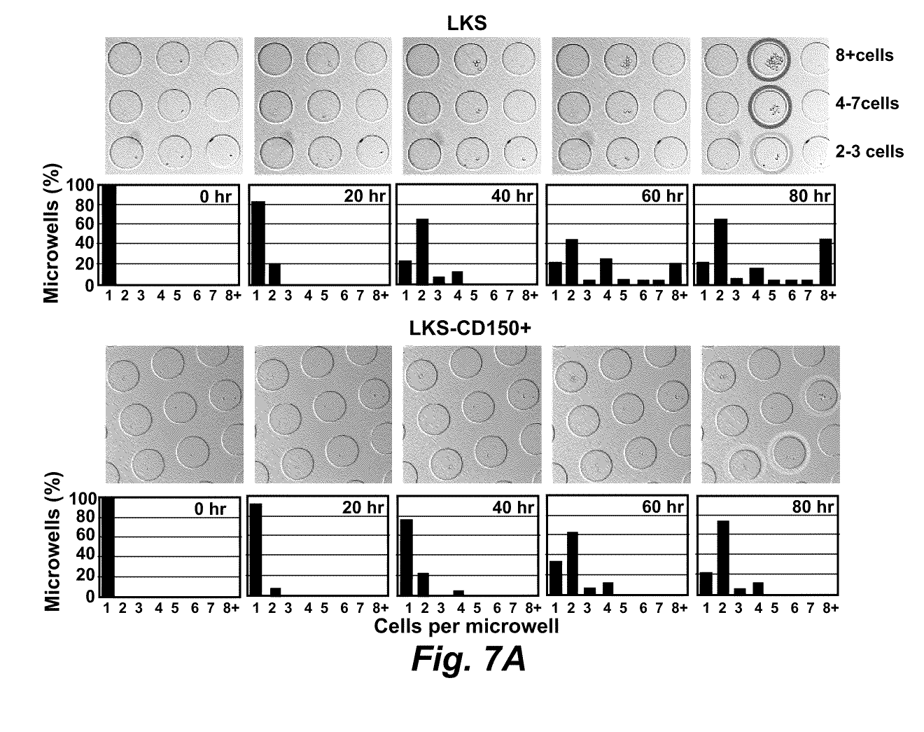

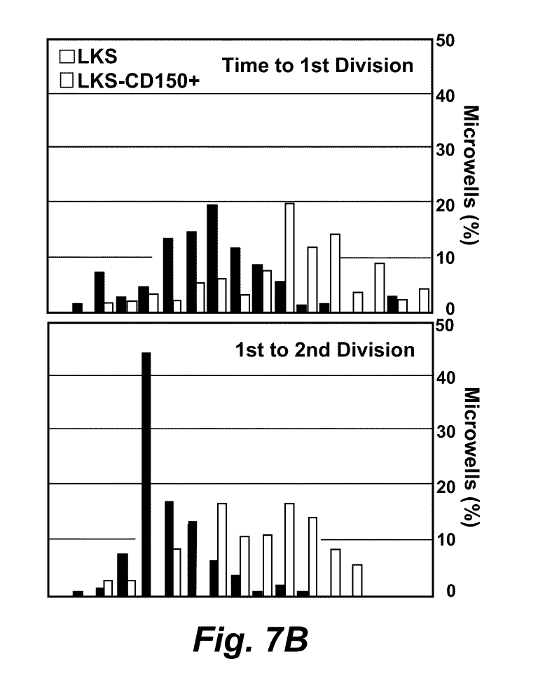

The role, not only of soluble factors but also of appropriately oriented membrane proteins, needs to be investigated without the complexity of co-culture. The present disclosure, therefore, encompasses methods of generating arrays of hydrogel microwells and a method for micro-contact printing that enables such as analyses of cell responses to a secreted and tethered membrane components normally provided by support cells within the niche. It will be understood, however, that the hydrogel compositions and the methods of their use in isolating a cell and providing appropriate soluble or tethered morphogens or growth factors may be readily adaptable for many types of cells including stem cells, progenitor cells and the like or non-stem cell lines. The gels and methods of the disclosure, for example, enable single HSC analyses with the arrays of topographically micro-patterned hydrogel microwells using cross-linked polyethylene glycol (PEG), a material that is both inert and transparent (FIGS. 6A-6C, 7A and 7B). In contrast to conventional tissue culture plastic or microwell arrays made of glass or rigid hydrophobic polymers (Chin et al., Biotechnology and Bioengineering 88, 399 (2004); Dykstra et al., Proc. Nat. Acad. Sci. U.S.A. 103, 8185 (2006)), the hydrogels of the present disclosure are soft (elastic modulus in the range of hundreds of Pascals (Lutolf & Hubbell, Nat Biotechnol 23: 47 (2005)) and have a high water content (>95%), more closely replicating the physicochemical properties of the in vivo niche and enhancing the viability of cultured cells.

The cell culture platform of the present disclosure may be fabricated from a soft and inert substrate that imbibes large amounts of water, thus approximating critical physicochemical aspects of the stem cell niche. The inertness of the polymeric substrate may preclude non-specific adsorption of proteins. However, proteins of interest can be specifically presented to cells by incorporating into the polymer network a heterofunctional PEG linker (tether) to which Protein A may be covalently conjugated, thereby allowing Fc-chimeric proteins to be selectively immobilized on the hydrogel surface. In this manner, specific Fc-chimers of proteins typically associated with cell-cell interactions in the niche can be tested without the complexity of co-culture.

While cell trapping and high-throughput single cell experimentation is afforded by several other microwell array systems (Revzin et al., Langmuir (2003) 19: 9855-9862; Koh et al., Biomedical Microdevices (2003) 5: 11-19; Dusseiller et al., Biomaterials (2005) 26: 5917-5925; Chin et al., Biotechnology and Bioengineering (2004) 88: 399-415; Mohr et al., Biomaterials, (2006), 27: 6032-6042; Khademhosseini et al., Biomaterials (2006) 27: 5968-5977; Karp et al., Lab on a Chip, (2007) 7: 786-794; Moeller et al., Biomaterials, (2008) 29: 752-763) these arrays were typically made with rigid and hydrophobic substrates than PEG hydrogels, such as PDMS or glass. Photopolymerized PEG had been used to fabricate hydrogel microwell arrays for the study of embryonic stem cell cultures but the selective tethering of proteins to this substrate was not been achieved.

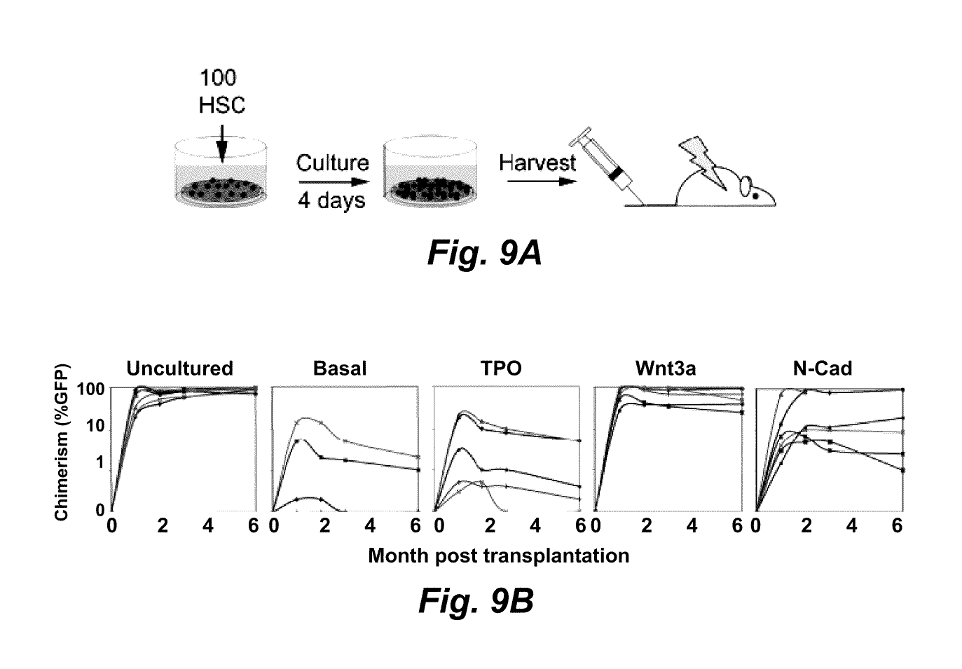

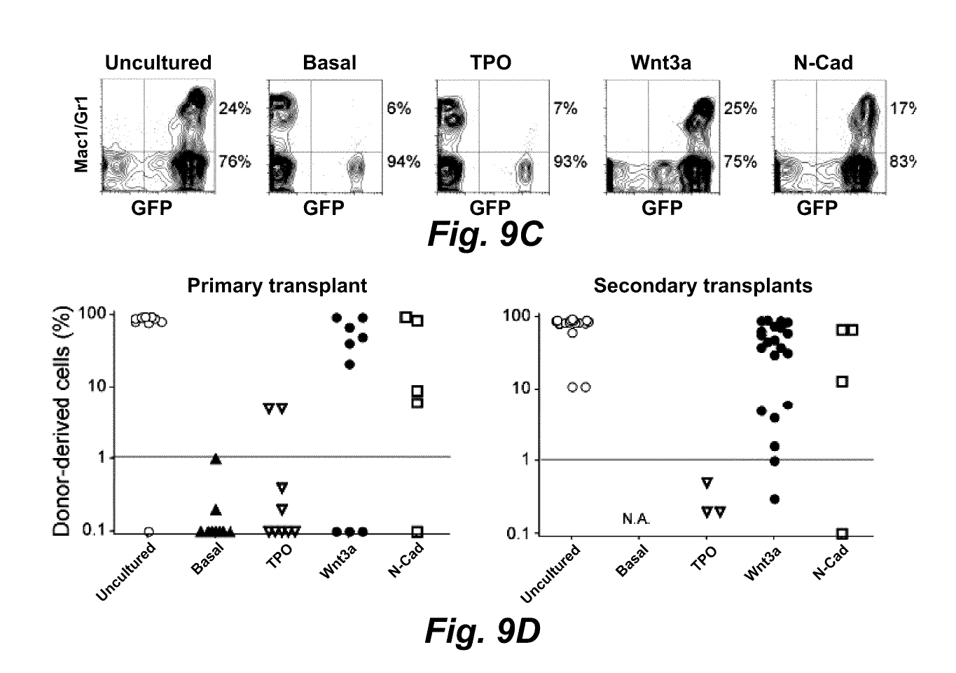

Embodiments of the hydrogel microwell array systems of the disclosure allow crosstalk of adult stem cells with their niche, and make possible the elucidation of the roles of factors that direct stem cell self-renewal or differentiation. For example, the single cell analyses using the hydrogel microwell platforms of the disclosure are in agreement with previous studies on the role of Wnt3a on HSC fate in tissue culture. As described in Example 9, below, soluble Wnt3a protein plays a role in the self-renewal of HSCs (Willert et al., Nature (2003) 423: 44S-452). When clones from 100 microwells were pooled and transplanted into lethally irradiated mice, reconstitution of the blood was observed (see FIGS. 9A-9D), a finding that could have been due to either persistence of the stem cell state or to self-renewal in these culture conditions. To distinguish between these two possibilities, doublets, i.e. cells that divided in culture once, were also transplanted. The finding that doublets reconstituted the blood upon transplantation is evidence that self-renewal of the stem cells had occurred. However, since the daughter cells were not separated for probing of their individual reconstitution potentials, whether Wnt3a induced asymmetric self-renewal divisions leading to stem cell maintenance or induced symmetric self-renewal divisions leading to stem cell expansion could not be distinguished. The absence of self-renewal upon transplantation of clones (more than two divisions) as shown in FIGS. 9A-9D for unmodified HSCs from wild type recipients, supports a role for Wnt3a in stem cell maintenance by asymmetric divisions rather than stem cell expansion.

Single cell analyses using the microwell systems of the disclosure showed that faster proliferation as seen with TPO correlates with a loss of HSC self-renewal function and shifts differentiation preferentially toward a lymphoid fate. A similar inhibition of self-renewal has been reported when HSCs were exposed to TPO in commercially available cytokine cocktails. Exposure of HSCs to TPO alone in microwells led to one dominant behavior: excessive proliferation and loss of stem cell potential. In a multi-factorial in vivo environment, however, TPO may have dual effects including a critical role in quiescence. It is contemplated that the microwell arrays of the disclosure will allow investigations into the interactions of two or more proteins, for example probing the effects of TPO together with Wnt3a or N-cadherin, simultaneously or sequentially, on stem cell function such as HSC function.

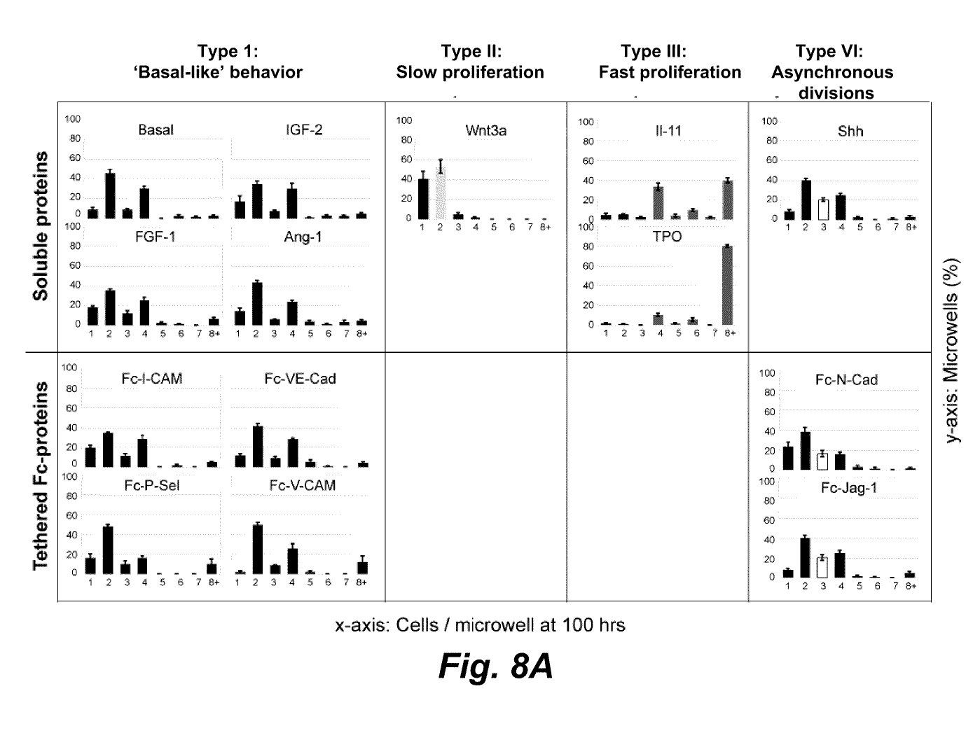

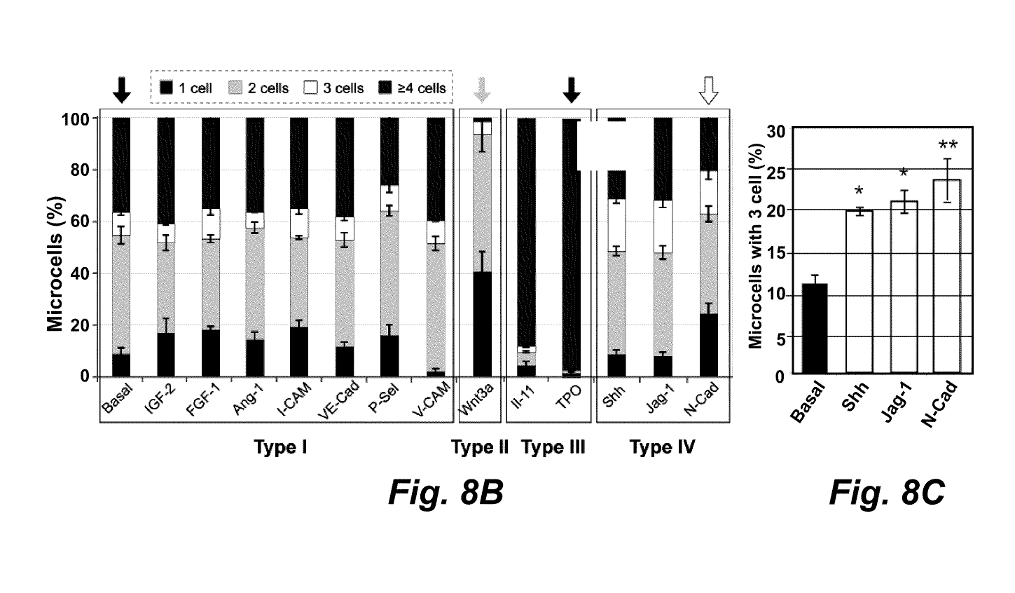

N-cadherin, when presented in vitro as immobilized on hydrogel surfaces that mimic physicochemical properties of the niche, as in the hydrogel microwell systems of the disclosure, can maintain single stem cells in a multipotent self-renewing state. The asynchrony in divisions of stem cell daughters (as shown in FIGS. 8A and 8B) would suggest the involvement of asymmetric divisions.

The data described in the Examples of the disclosure indicate that (1) the kinetic behavior of rare populations of adult stem cells can be systematically studied and manipulated at the single cell level using arrays of hydrogel microwells in conjunction with time-lapse microscopy, (2) cell-cell interactions can be mimicked without the complexity of co-culture by tethering and, therefore, properly orienting membrane niche proteins, (3) exposure of single cells for 4-7 days to single extrinsic cues typical of the niche have profound effects on stem cell function in vitro enabling reconstitution of the blood in vivo, (4) division kinetics (slow or asynchronous proliferation) of single HSCs in response to single proteins (such as Wnt3a and N-cadherin, respectively) correlates with in vivo stem cell function. The identification of specific molecules that may influence HSC maintenance by self-renewal, and ultimately HSC expansion without genetic manipulation, are important in overcoming the limitation of cell numbers currently available for transplantation

One aspect of the disclosure is a system for isolating or culturing a eukaryotic cell, the system comprising a hydrogel film comprising a cross-linked polymeric composition having the characteristic of hydrating to form a hydrogel and having a topographical feature or a plurality of topographical features, wherein each topographical feature may have a surface capable of receiving and immobilizing at least one biomolecule species thereon.

In embodiments of this aspect of the disclosure, the hydrogel film may be hydrated as a hydrogel.

In embodiments of this aspect of the disclosure, the system may further comprise at least one biomolecule species immobilized to the cross-linked polymeric composition. In these embodiments of the disclosure, the biomolecule species may be selected from the group embodiments of this aspect of the disclosure, the biomolecule may be selected from the group consisting of: Wnt3a, N-cadherin, thrombopoietin, erythropoietin, granulocyte-macrophage colony stimulating factor, granulocyte colony stimulating factor, macrophage colony stimulating factor, thrombopoietin, stem cell factor, interleukin-1, interleukin-2, interleukin-3, interleukin-6, interleukin-7, interleukin-15, Flt3L, leukemia inhibitory factor, insulin-like growth factor, insulin, and recombinant insulin.

In embodiments of the system of the disclosure, the cross-linked polymeric composition may be selected from the group consisting of: a poly(ethylene glycol), a polyaliphatic polyurethane, a polyether polyurethane, a polyester polyurethane, a polyethylene copolymer, a polyamide, a polyvinyl alcohol, a polypropylene glycol, a polytetramethylene oxide, a polyvinyl pyrrolidone, a polyacrylamide, a poly(hydroxyethyl acrylate), and a poly(hydroxyethyl methacrylate)

In embodiments of the disclosure, wherein the cross-linked polymeric composition may be formed from at least two precursor compounds in a ratio whereby, when the precursors are cross-linked to form the cross-linked polymeric composition, a surface of a topographical feature is capable of immobilizing a polypeptide or a tether thereto.

In embodiments of the systems of the disclosure the cross-linked polymer composition may be synthesized from at least two precursor compounds wherein one precursor compound comprises n nucleophilic groups, and a second precursor compound comprises m electrophilic groups, wherein n and m are each at least 2 and the sum (n+m) is at least five.

In one embodiment, the cross-linked polymeric composition is synthesized from at least two precursor components using a Michael-type addition reaction.

In another embodiment the n nucleophilic groups may be thiol groups.

In yet another embodiment of the disclosure, the m electrophilic groups may be conjugated unsaturated groups.

In the embodiments of the disclosure, the plurality of topographical features may be an array of microwells.

In the embodiments of the disclosure, a surface of a topographical feature may comprise a reactive functional group of the cross-linked polymeric composition, wherein the reactive functional group can be capable of binding to the biomolecule species desired to be immobilized on the surface of the topographical feature. In these embodiments, the reactive functional group may be selected from the group consisting of: a succinimidyl active ester, an aldehyde, a thiol, and a thiol-selective group.

In other embodiments of the system of the disclosure, a topographical feature may have a tether immobilized thereon, wherein the tether may be capable of selectively binding to the biomolecule species desired to be immobilized on the surface of the topographical feature.

In embodiments of the disclosure, the tether may be selected from the group consisting of: a peptide, a polypeptide, and a non-peptide linker. In some embodiments, the tether may be selected from the group consisting of: a heterofunctional PEG, Protein A, Protein G, an immunoglobulin, streptavidin, neutravidin, biotin, a linker capable of forming a complex with a metal ion, and a transglutaminase substrate. In other embodiments, the tether can be Protein A or Protein G, and the biomolecule species bound thereto is a polypeptide comprising an immunoglobulin Fc region and a region capable of interacting with a cell disposed in the topographical feature.

In the various embodiments of the disclosure, the hydrogel film may also be disposed in a well of a multi-well tissue culture plate.

Another aspect of the disclosure are microcontact printing methods of preparing a hydrogel, comprising: (a) providing a template comprising a negative topographical feature or a plurality of negative topographical features, wherein each negative topographical feature defines a topographical feature desired to be formed in a hydrogel, and wherein the negative topographical feature or features has on the surface thereof a biomolecule species desired to be transferred to a hydrogel, a tether capable of selectively binding to a biomolecule species, or a combination thereof; (b) delivering to the template a hydrogel polymer precursor composition; (c) polymerizing the hydrogel cross-linked polymeric composition to form a hydrogel film; and (d) removing the hydrogel film from the template, thereby transferring the biomolecule species, the tether, or a combination thereof, to a surface of the topographical feature molded in the hydrogel film.

In embodiments of this aspect of the disclosure, each topographical feature molded in the hydrogel may be a microwell.

In other embodiments of the disclosure, the method may further comprise hydrating the hydrogel film, thereby forming a hydrogel.

In the embodiments of this aspect of the disclosure, the biomolecule species may be selected from the group consisting of: a polypeptide, a peptide, an oligonucleotide, and a small molecule. In these embodiments, the biomolecule may be selected from the group consisting of: Wnt3a, N-cadherin, thrombopoietin, erythropoietin, granulocyte-macrophage colony stimulating factor, granulocyte colony stimulating factor, macrophage colony stimulating factor, thrombopoietin, stem cell factor, interleukin-1, interleukin-2, interleukin-3, interleukin-6, interleukin-7, interleukin-15, Flt3L, leukemia inhibitory factor, insulin-like growth factor, insulin, and recombinant insulin, or a combination thereof.

In the various embodiments of this aspect of the disclosure, if a tether is transferred from the template to a surface of a topographical feature, the method further may comprise delivering to the topographical feature of the hydrogel a composition that may comprise a biomolecule species desired to be immobilized on the surface of the microwells, thereby selectively binding the biomolecule species to the tether and immobilizing the biomolecule species to the surface of the topographical feature.

In other embodiments, a surface of a topographical feature or of a plurality of topographical features may comprise a reactive functional group of the cross-linked polymeric composition, wherein the reactive functional group can be capable of binding to the biomolecule species desired to be immobilized on the surface of the topographical feature or plurality of topographical features.

In embodiments of the disclosure, the reactive functional group may be selected from the group consisting of: a succinimidyl active ester, an aldehyde, a thiol and a thiol-selective group.

In embodiments of the disclosure, a surface of a topographical feature may have a tether immobilized thereon, wherein the tether may be capable of selectively binding to the biomolecule species desired to be immobilized on the surface of the topographical feature. In these embodiments, the tether may be selected from the group consisting of: a peptide, a polypeptide, and a non-peptide linker. In these embodiments, the tether may be selected from the group consisting of: a heterofunctional PEG, Protein A, Protein G, an immunoglobulin, streptavidin, neutravidin, biotin, a linker capable of forming a complex with a metal ion, and a transglutaminase substrate. In various embodiments, the tether is Protein A or Protein G, and the biomolecule species bound thereto may comprise an immunoglobulin Fc region and a region capable of interacting with a cell disposed in the topographical feature.

In embodiments of this aspect of the disclosure, the hydrogel polymer precursor composition may comprise at least two precursor compounds in a ratio whereby when the precursors are cross-linked to form the polymer the surface of the microwells of the microwell array is capable of immobilizing a polypeptide or a tether thereto.

In the various embodiments of this aspect of the disclosure, the hydrogel film may be disposed in a well of a multi-well tissue culture plate.

Another aspect of the disclosure is a method of isolating individual cells from a population of cells, wherein the methods may comprise: (a) providing a hydrogel system disposed in a well of a multi-well tissue culture plate, wherein the hydrogel comprises a hydrated cross-linked polymer having an array of topographical features indented therein, and wherein a surface of each of the topographical features has at least one biomolecule species immobilized thereon; (b) delivering a cell suspension of isolated cells to the well of the multi-well plate, whereby the cells of the suspension descend under gravity into the multiplicity of microwells, and wherein the cell density of the cell suspension is adjusted whereby at least one well of the multiplicity of wells receives a single cell; and (c) incubating the hydrogel under conditions favorable for proliferation of the cells.

In embodiments of this aspect of the disclosure, the topographical feature may be a microwell.

In the various embodiments of the methods of this aspect of the disclosure, the cell suspension may comprise a population of stem cells. In these embodiments, the population of stem cells may be selected from the group consisting of: hematopoietic stem cells, hematopoietic progenitor cells, adult stem cells, embryonic stem cells, and cancer stem cells.

In various embodiments of this aspect of the disclosure, a biomolecule species may be immobilized by a tether to a surface of a topographical feature.

In the embodiments of the disclosure, the at least one biomolecule species may be selected from the group consisting of: a polypeptide, a peptide, an oligonucleotide, and a small molecule. In these embodiments, the biomolecule may be selected from the group consisting of: Wnt3a, N-cadherin, thrombopoietin, erythropoietin, granulocyte-macrophage colony stimulating factor, granulocyte colony stimulating factor, macrophage colony stimulating factor, thrombopoietin, stem cell factor, interleukin-1, interleukin-2, interleukin-3, interleukin-6, interleukin-7, interleukin-15, Flt3L, leukemia inhibitory factor, insulin-like growth factor, insulin, and recombinant insulin, or a combination thereof.

In embodiments of this aspect of the disclosure, a surface of a topographical feature may comprise a reactive functional group of the cross-linked polymeric composition, wherein the reactive functional group can be capable of binding to the biomolecule species desired to be immobilized on the surface of the topographical feature. In these embodiments, the reactive functional group may be selected from the group consisting of: a succinimidyl active ester, an aldehyde, a thiol and a thiol-selective group.

In embodiments of the disclosure, the tether may be selected from the group consisting of: a peptide, a polypeptide, and a non-peptide linker. In these embodiments, the tether may be selected from the group consisting of: a heterofunctional PEG, Protein A, Protein G, an immunoglobulin, streptavidin, neutravidin, biotin, a linker capable of forming a complex with a metal ion, and a transglutaminase substrate. In other embodiments, the tether may be Protein A or Protein G, and the biomolecule species bound thereto may comprise an immunoglobulin Fc region and a region capable of interacting with a cell disposed in the topographical features.

Still yet another aspect of the disclosure is a method for determining the proliferative outcome of transplanting a stem cell into a recipient host, comprising; (a) delivering a population of cells to a plurality of microwells indented in a hydrogel, wherein an interior surface of each microwell of the plurality of microwells has a biomolecule species immobilized thereon, and wherein at least some of the microwells of the multiplicity of microwells may receive a single cell from the population of cells; (b) monitoring the proliferation of the isolated single cells by time-lapse photography; (c) correlating the proliferation of the cells to the proliferative outcome of a stem cell transplanted into a recipient host; and (d) identifying those cells in a microwell having the characteristic of regenerating when transplanted into a recipient host.

In one embodiment of this aspect of the disclosure, the stem cell is a hematopoietic stem cell.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and the present disclosure and protected by the following claims.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### EXAMPLES

### Example 1

### Isolation and Purification of Hematopoietic Stem/Progenitor Cells by Flow Cytometry

Bone marrow donors were 8- to 12-week-old GFP⁺ C57BL/6-Ly5.1 mice. After isolating the bone of the hind legs, the bone marrow of the femurs and tibias was extensively flushed with several mls of FACS buffer consisting of 1×PBS pH 7.4 containing 12.5% fetal bovine serum (FBS) (Omega Scientific, USA) and 2 mM EDTA. The cell suspension was filtered through a 70 μm nylon cell strainer (BD Falcon, USA), filled to 40 mls with the above FACS buffer, supplemented with 10 ml FBS, and centrifuged for 10 min at 1400 rpm. The remaining pellet was resuspended in 5 ml red blood cell (RBC) lysis buffer, incubated on ice for 5 min, filled to 40 ml with FACS buffer, supplemented with 10 ml FBS and centrifuged for 10 min at 1400 rpm and again resuspended in 1 ml FACS buffer. Lin⁻c-kit⁺Sca1⁺(LKS) cells, and a subpopulation of LKS expressing the SLAM receptor CD150 (LKS-CD150+) cells were isolated. A mouse lineage panel (BD Biosciences, USA, used according to manufacturer's instructions) was used to stain differentiated cells. In short, 3 μl Fc-block (anti-CD16/CD32 BD, Bioscience) and 20 μl of each lineage panel antibody (anti-CD3e biotin, anti-CD45R/B220 biotin, anti-CD11b biotin, anti-Ly-6G biotin and anti-TER-119 biotin) were added to the cell suspension and incubated on ice for 20 min. Then, 10 mls FACS buffer were added to the cell suspension and centrifuged at 1400 rpm for 10 min.

The remaining cell pellet was resuspended in 900 μl FACS buffer and stained by adding 100 μl streptavidin magnetic microbeads (Miltenyi Biotech, Germany), 5 μl anti-c-Kit-PE/Cy7 (eBioScience, USA), 10 μl anti-Sca1-PE (BD Bioscience, USA), 10 μL anti-CD150-APC (Bio Legend, USA) and 5 μl Texas Red-Streptavidin (Molecular Probes, USA). After an incubation time of 30 min at 4° C. under gentle shaking using a rotating plate, 10 ml FACS buffer were added and centrifuged at 1400 rpm for 10 min. The suspension was resuspended in 1 ml FACS buffer and separated using a MidiMACS magnetic column (Miltenyi Biotech, Germany). The eluted cell suspension was centrifuged at 1400 rpm for 10 min and resuspended in 3 ml FACS buffer, 3 μl propidium iodide (PI) was added.

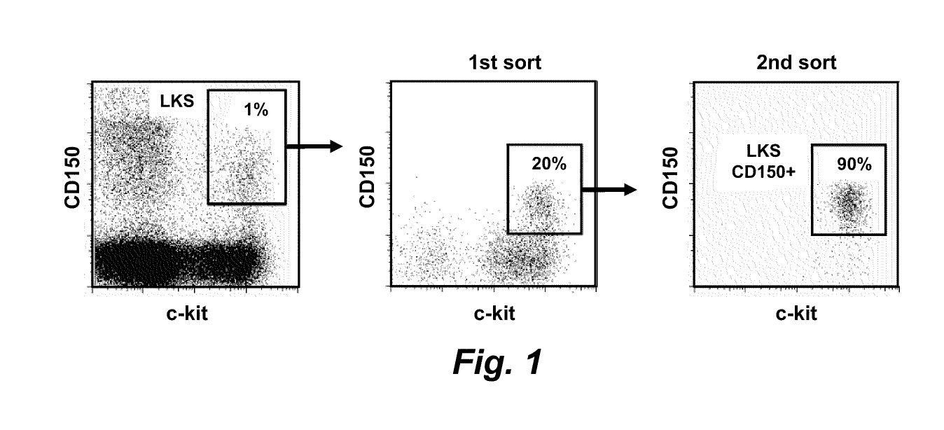

The lineage-depleted cell population was further separated by flow cytometry on a Vantage SE FACS instrument (BD Bioscience, USA). Single viable (propidium iodide negative) Lin⁻c-kit⁺Sca1⁺CD150⁺ cells were triple sorted using the gates shown in the FACS plots of FIG. 1 and directly deposited in microwell arrays, as described in Example 4 below. FACS data were plotted using FlowJo (TreeStar Inc., USA).

### Example 2

### Long-Term Reconstitution Assays

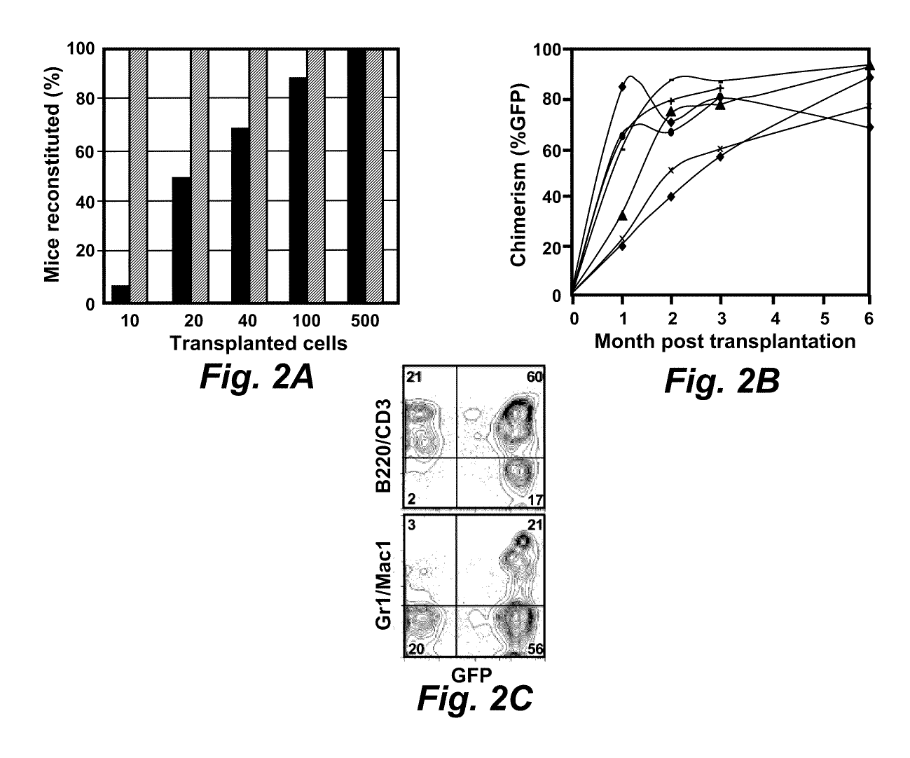

Referring now to FIG. 2, to assess the self-renewal potential, long-term blood reconstitution assays were conducted. 10, 20, 100 or 500 GFP⁺ cells (C57BL/6, Ly5.1) of the LKS or LKS-CD150⁺ population were transplanted per animal, together with 5×10⁵ GFP⁻Sca1⁻CD150⁻ bone marrow ‘helper’ cells (C57BL/6, Ly5.1) into lethally irradiated wild-type host mice (C57BL/6, Ly5.2) as described by Corbel et al., Nat. Med., (2003) 9: 1528-1532, incorporated herein by reference in its entirety. All transplant recipients were Ly5-congenic, and a split dose irradiation, i.e. two sequential doses of 4.8Gy, was used.

After sorting the donor population, the number of cells to be injected per mouse was re-sorted into individual wells of a 96-well plate containing 5×10⁵ “helpers” incapable of long-term reconstitution (C57BL/6, Ly5.1).

The contents of individual wells were injected into the tail veins of individual lethally irradiated recipients. Reconstitution was measured by assessment of GFP⁺Ly5.1⁺ cells in the CD45 gated peripheral blood from retro-orbital bleeding 4, 8, 12, and 24 weeks after transplantation. Blood was subjected to RBC lysis with ammonium chloride, and white blood cells were stained with directly conjugated antibodies to CD45.2 (104, FITC), B220 (6B2), Mac-1 (M1/70), CD3 (KT31.1), and Gr-1 (8C5) to monitor engraftment.

### Example 3

### Fabrication of Hydrogel Microwell Arrays for High-Throughput Analysis of Single Stem Cell Behavior

(a) Poly(ethylene glycol) (PEG): 8arm-PEG-OH (mol. wt. 4×10⁴ g/mol) and linear PEG-(SH)₂ (mol. wt. 3.4×10³ g/mol, 100% substitution) was used. Divinyl sulfone was purchased from Aldrich (Buchs, Switzerland). 8arm-PEG-vinylsulfones (8arm-PEG-VS) were produced and characterized as described by Lutolf & Hubbell, Biomacromolecules (2003) 4: 713-722, incorporated herein by reference in its entirety. The final product was dried under vacuum and stored under argon at −20° C.

The degree of end group conversion, confirmed with 1H NMR (CDCl₃): 3.6 ppm (PEG backbone), 6.1 ppm (d, 1H, ═CH₂), 6.4 ppm (d, 1H, ═CH₂), and 6.8 ppm (dd, 1H, —SO₂CH═), was found to be 87%.

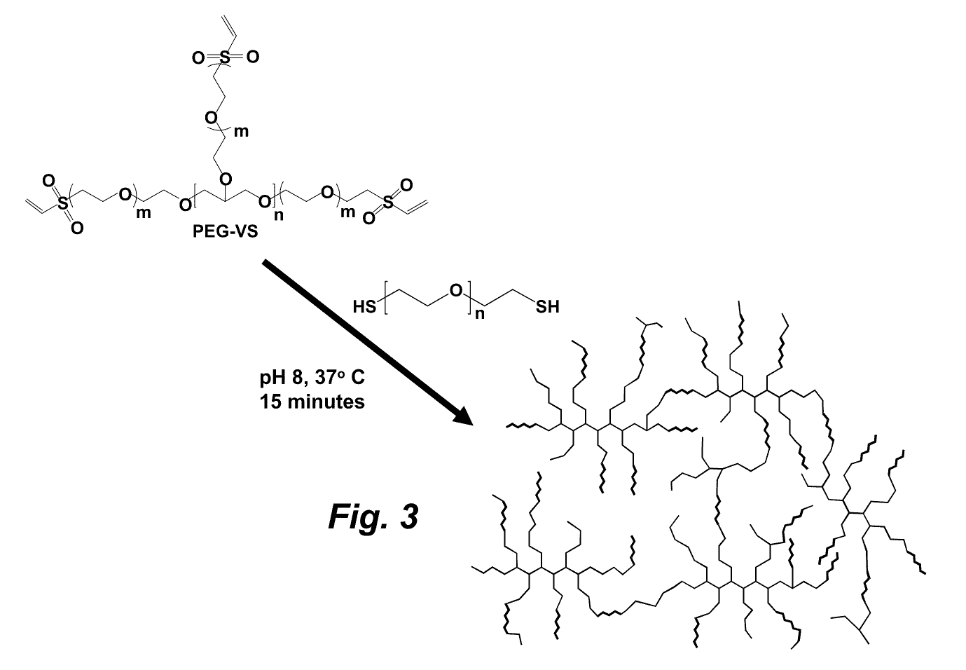

(b) Gelation of PEG precursors: Referring now to FIG. 3, chemistry described by Lutolf et al., Advanced Materials 15, 888 (2003), incorporated herein by reference in its entirety, was modified to form hydrogel films from the above PEG precursors in stoichiometrically balanced amounts. Both precursors were dissolved at a solid concentration of 10% (w/v) in 0.3 M triethanolamine (8-arm-PEG-VS), and in ultra pure water (PEG-(SH)₂), respectively, and mixed to form cross-linked gel networks by Michael-type addition.

To avoid batch-to-batch variability, each precursor solution was prepared in large quantities (of about 2.5 ml), filter sterilized (0.22 μm) and aliquoted in amounts for the synthesis of approximately 250 μl PEG hydrogel.

(c) Hydrogel microwell array formation: Referring to FIG. 4, hydrogel microwell arrays were fabricated by a multistep soft lithography process. PDMS microwell array replication masters of the size of an entire Si wafer were obtained. Prior to PEG gel casting, the PDMS master was cut to a size matching a desired well-format (96-, 48- or 24-well), thoroughly cleaned, and then modified with a surface layer of ₁H,₁H,₂H,₂H-perfluorodecyltrichlorosilane (Oakwood Chemicals, USA). Immediately after mixing of the above precursors in an Eppendorf tube, the PEG precursor solutions (approximately 80 μl for the 24-well size) was pipetted on the PDMS surface positioned on a hydrophobic glass slide (precoated with SIGMACOTE™, Sigma, USA).

Appropriate spacers, each 0.7 mm thicker than the thickness of the PDMS master were placed at both ends of the glass slide and a second hydrophobic slide was placed on top. The two slides were fixed with binder clips on both ends, ensuring an optimal wetting of the PDMS microstructures with the precursor solution. Curing of the gel network was conducted for 30 min at 37° C. in a humidified incubator. The PEG-based hydrogel microwell arrays thus formed were peeled off using a pair of blunt forceps, washed at least 4×15 min with 4 ml PBS, and swollen overnight in PBS. As shown in FIG. 5, before cell culture, the swollen PEG hydrogel microwell arrays were fixed on the bottom of plastic wells of a desired well plate using the above gel precursor solution as efficient ‘glue’, and the arrays were equilibrated at 37° C. in cell culture medium.

### Example 4

### Hematopoietic Stem Cell Culture

LKS-CD150⁺ cells were cultured under sterile conditions in a serum-free environment using Stemline II hematopoietic expansion medium (Sigma, USA) supplemented with 100 ng/ml Stem Cell Factor (SCF) and 2 ng/ml Flt-3 ligand in 10% CO₂ at 37° C. in a humidified incubator. In a typical experiment, about 300 individual LKS-CD150⁺ cells were seeded per well of a 96-well plate containing a total of about 400 microwells in 200 μl of medium (1000 to 2000 cells per well of a 24-well plate containing about 4000 microwells in 1 ml of medium when micromanipulation was to be performed). After 1 hour during which individual cells randomly sedimented onto the bottom of microwells, the plate was transferred to the incubator of the microscope and further cultured under the same sterile conditions for at least four days.

### Selection of Putative Soluble HSC Regulatory Proteins.

The soluble proteins listed in Table 1 below were tested for their effect on HSC fate. These factors were added to the above basal medium at the specified concentrations selected based on previous reports. 10% FBS served positive control. For 7-day cultures, fresh medium and factors were added at day 4.

TABLE 1
Tested soluble HSC regulatory proteins

| Candidate | | | | | |
|---|---|---|---|---|---|
| Protein | Suggested niche role | Niche | Source | Conc. | |
| Wnt3a | HSC self-renewal | Endos- | mouse | 100 | ng/ml |
| | | teal | | | |
| IL-11 | Cytokines stimulating | NA | mouse | 20 | ng/ml |
| | HSC expansion | | | | |
| FGF-1 | Maintenance of function | NA | human | 10 | ng/ml |
| | HSC in vitro | | | | |
| TPO | Maintenance of HSC | NA | mouse | 100 | ng/ml |
| | activity and self-renewal | | | | |
| IGF-2 | Stimulate HSC expansion | NA | mouse | 20 | ng/ml |
| Ang-1 | HSC quiescence and cell | NA | human | 1 | ug/ml |
| | cycle regulation | | | | |
| Shh | Proliferation of HSC | NA | mouse | 100 | ng/ml |

### Example 5

### Time-Lapse Microscopy and Image Analysis

LKS⁻CD150⁺ cells were directly sorted into multi-well plate wells containing the microwell surfaces, as shown in FIG. 5. The plate was then transferred to the environmental chamber of an inverted microscope (Zeiss Axiovert 200M) equipped with a motorized stage. After cells were randomly distributed and trapped in microwells by gravitational sedimentation, the XYZ stage was programmed to repeatedly raster across the microwell array surface, acquiring phase contrast images at 10× (in some cases 20×) magnification of multiple locations in defined time intervals for a period of up to 7 days, or as specified as shown, for example, in FIG. 5.

The number of independent regions per sample was chosen so as to capture at least 100 single live cells in microwells per condition at the start of the experiment. The resulting images of such a time-lapse experiment were then automatically compiled into a stack (library) using the Volocity software (Improvision). Cells were scored as dead when they ceased to move on the microwell surface. To confirm the death read-out, cells were stained by adding propidium iodide (at a 1:10 ratio) in PBS.

To assure highest accuracy in determining individual cell proliferation kinetics, the number of cells per microwell was manually counted for each time point. However, scoring of time-lapse movies was facilitated by a Matlab program designed to take advantage of high-throughput automated image analysis while maintaining the high accuracy of manual counting. Image stacks were segmented into individual microwell stacks using a binary mask generated from graphical user input and the known periodicity of the microwell array. The number of cells in each microwell at every time point was first determined using a customized cell segmentation algorithm. A Matlab script was used to manually review all microwells found to contain at least one cell and errors arising from automated analysis were manually corrected. The raw data containing the cell count and the region location was then compiled on an Excel spreadsheet for further statistical analysis of the growth kinetics of individual live cells.

Growth kinetic data were derived from a quantification of the extent of proliferation, or total cell number, at time intervals for each microwell. Since the microwell platform in conjunction with time-lapse microscopy was designed to perform high-throughput experiments, a means of facilitating cell counting was required. To obtain proliferation data at a clonal level and count cells in individual microwells, a customized, semi-automated cell counting program (Matlab, (Mathworks Company)) was used. Starting with a master image containing all 400 microwells within an array, edge detection was used to locate all microwells and then segment each microwell into its own image, yielding 400 separate images per array. For each microwell containing a single cell, a series of images corresponding to that microwell was generated automatically at each time point (every 24 hours). This program allowed rapid selection of the microwells for analysis and automatically visualized successive frames of time-lapse movies of the same microwell on the computer screen, enabling rapid and precise visual evaluation and recording of cell division in an annotated Excel format.

### Example 6

### In Situ Patterning of Biomolecules on Microstructured Gels Via ‘Reactive Microcontact Printing’

Referring now to FIGS. 6A-6D, to control both topography and the localized presentation of putative extracellular and transmembrane HSC regulatory proteins on gel microwell arrays, protein immobilization was restricted to selected areas on the gel surface via a hydrogel microfabrication process termed ‘reactive microcontact printing’.

PDMS replication masters prepared as described in Example 3, above, were first ‘inked’ using PEG-modified Protein A (a protein that can strongly bind engineered Fc-chimeric proteins) to adsorb it just on the tips of positive template features such as pillars. For this purpose, Protein A was pre-reacted for 30 min at room temperature with a 10-fold molar excess of a heterofunctional NHS-PEG-VS PEG linker (Nektar, Huntsville, Ala., USA). This allowed the free VS-groups to be covalently attached to the gel surface in the next step. Hydrogel microwell casting was then conducted, as described in Example 3 above, on this Protein A-adsorbed template, transferring and locally covalently immobilizing Protein A from the PDMS surface to the forming, microstructured gel matrix. Subsequently, Protein A-modified PEG hydrogel microwells were incubated with 400 μl (in the case of a hydrogel microwell array being placed in the well of a 24-well plate) of a solution of a desired Fc-chimeric protein (at 10 μg/ml in PBS). After an incubation time of 1 hour at 37° C. in a humidified incubator, the microwell samples were washed 4×15 min with PBS to remove non-immobilized Fc-chimeric proteins. Non-specific protein adsorption on the sample was minimized by incubating with 4 ml of a solution of 1% BSA (w/v) in PBS (0.22 μm filter sterilized) for 1 hour at room temperature before the immobilization of the Fc-chimeric proteins.

(a) Selection of putative tethered HSC regulatory proteins: Fc-chimeric proteins listed in Table 3 were tested for their effect on HSC fate.

TABLE 3
Tested transmembrane (Fc-chimeric)
HSC niche regulatory proteins

| Candidate | | | |
|---|---|---|---|
| Protein | Suggested role in the niche | Niche | Source |
| Jagged-1 | Notch ligand self-renewal and | Endosteal | rat |
| | clonal expansion | | |
| N-cadherin | Homotypic interaction anchorage/ | Endosteal | human |
| | quiescence role | | |
| VE-cadherin | Interaction of megakaryocytes | Vascular | human |
| | with sinusoidal bone marrow | | |
| | endothelial cells (BMEC); | | |
| | promotion of megakaryocyte | | |
| | maturation | | |
| ICAM-1 | Physical contact with osteoblast | | mouse |
| | for HSC survival | | |
| VCAM-1 | Heterotypic interaction with | Vascular | mouse |
| | VLA4 | | |
| E-Selectin | HSC adhesion to osteoblasts; | Vascular | mouse |
| | Homing and engraftment into the | | |
| | niche; differentiation into | | |
| | myeloid progenitors | | |
| P-Selectin | Homing and engraftment into the | Vascular | mouse |
| | niche; expansion of hematopoietic | | |
| | progenitors | | |

(b) Qualitative assessment of efficiency of microwell protein tethering using confocal laser scanning microscopy: Referring now to FIG. 6D, confocal laser scanning microscopy was used to test the extent, uniformity, and stability of the microcontact printing process. In a first step, a human IgG Fc-fragment (BiosPacific, USA) was utilized as a model protein binding to Protein A. The Fc-fragment was labeled with Alexa Fluor 488 using a Monoclonal Antibody Labeling Kit (Molecular Probes, USA). Conjugation reaction, purification and determination of the degree of labeling were done according to the manufacturer's protocol.

Spectral absorbance was measured using a Nanoprop ND-1000 spectrophotometer (Nanoprop Technologies, USA). The Fc-fragment was conjugated to the Protein A-tethered microwell bottom as described above. Images were acquired using a LSM 510 META confocal laser scanning microscope (Zeiss, Germany). Typically, z-stacks were acquired with a constant slice thickness of 1.5-2 μm, reconstructing a cross section profile of approximately 150 μm. Cross section analysis, 3D-reconstructions and image processing were done using Volocity (Improvision, USA) and Photoshop CS (Adobe, USA).

Tethering of the selected Fc-chimeric proteins listed in Table 3 was also assessed via immunostaining. For example, N-cadherin-functionalized PEG hydrogel microwells were synthesized as described. After blocking in 4 ml PBS containing 1% BSA for 1 hour at room temperature, the samples were washed 4× for 15 min in 4 ml PBS. The hydrogels were then incubated for 1 hour at room temperature with 1 ml of a solution of mouse monoclonal anti-N-cadherin IgG (BD Biosciences, USA) at 1:1000 in PBS containing 3% goat serum, followed by subsequent washing for 4× for 15 min in 4 ml PBS. The secondary antibody incubation was conducted for 1 hour at room temperature using 1 ml of an Alexa Fluor 488 labeled goat anti-mouse IgG (Invitrogen, USA) dissolved 1:500 in PBS plus 3% goat serum. Afterwards the samples were washed 4× for 15 min in 4 ml PBS and imaged via confocal microscopy as described above.

### Example 7

### Stem Cells Exhibit Slower In Vitro Division Kinetics than Multipotent Progenitors

The extent of division of the stem cell enriched LKS-CD150+ cell population would be reduced, as the cells would divide more slowly in culture than would LKS cells. To test this hypothesis the division kinetics of each population were assessed by seeding 300 single cells per well of a 96-well plate containing a total of 400 microwells (FIG. 5). Single cells randomly sedimented to the bottoms of microwells within minutes. Microwells with multiple cells at the onset of the experiment were eliminated retrospectively from the analysis. At least 100 single cells of both populations were tracked by automated time-lapse microscopy and the kinetic proliferation profiles quantified as the distribution of numbers of HSC progeny generated per microwell as a function of time. When cultured in a basal serum-free culture medium supplemented with only stem cell factor (SCF, or c-kit ligand; 100 ng/ml) and Flt-3 ligand (2 ng/ml), cells of the two phenotypes exhibited marked differences in division kinetics (FIGS. 7A and 7B). LKS proliferated rapidly compared to LKS-CD150+, as shown the digital images of representative time-lapse analyses and a quantification of the distribution of cells per microwell at various time points (FIG. 7A).

The progeny of 100 single GFP⁺ CD150⁺ HSCs grown for 7 days in a medium containing stem cell factor (SCF) and Flt-3 ligand were tested for stem cell function by the classic assay of transplantation into lethally irradiated mice depleted of endogenous stem cells. The low reconstitution of the blood indicated that essential niche factors were lacking.

LKS⁻CD150⁺ cells grown in PEG hydrogel microwells were tested under basal conditions described above, with no additional factors that would support HSC function. The progeny of 100 single GFP⁺ cells grown for 7 days in microwells were tested for stem cell function by transplantation of the cultured cells into lethally irradiated mice. In contrast to freshly isolated LKS⁻CD150⁺ cells, which led to high peripheral blood reconstitution upon transplantation in mice (5/5), none of the mice injected with cells from microwells exhibited reconstitution (0/5), indicating that essential niche factors were lacking in the culture system. These data provided the impetus for the systematic studies of individual soluble and tethered factors implicated in the HSC niche on HSC function.

### Example 8

To replicate HSC-niche interactions in vitro in a near-physiologic fashion, potential morphogen or growth factor proteins were spatially patterned and immobilized onto the hydrogel microwell matrices as shown in FIGS. 6A-6D. Protein tethering was achieved by attaching a heterofunctional PEG linker, or tether, to a protein of interest and then cross-linking this complex into the gel network. To ensure site-selectivity in protein immobilization, engineered Fc-chimeric proteins that could be linked via binding to an intermediate auxiliary protein, Protein A that contains four high-affinity binding sites (Kₛ=10/mole) specific for the Fc-region of human, mouse and rabbit immunoglobulins.

Accordingly, to specifically functionalize gels and immobilize proteins only at the bottom of microwells, rather than homogeneously distributing proteins across the entire array surface (bulk modification), the microwell fabrication process shown in FIG. 4 was augmented by adding a reactive microcontact printing step of the disclosure (FIG. 6B). Thus, PEG-functionalized Protein A was adsorbed onto the extended micropillars of the PDMS stamp (FIG. 6B, steps 1 and 2) and the hydrogel was then polymerized against the PDMS (FIG. 6B, steps 3 and 4), transferring both the topographical feature pattern and protein pattern onto the gel surface.

Selective modification of microwells with Fc-chimeric adhesion proteins such as V-CAM ensured efficient confinement and tracking of HSCs over long culture periods. This contrasts with microwell arrays where the bulk of the surface was modified rather than just the bottoms of the wells alone. In the latter case, the cells escaped from the microwells within a few hours.

Immunofluorescence microscopy revealed that microcontact printed proteins, such as a BSA-FITC model protein were localized at the bottom of the microwells, as shown in FIG. 6C. When Protein A was used as the linker or tether, Fc-chimeric proteins such as N-cadherin (N-cad) were also shown via immunostaining to be effectively immobilized, as shown in FIG. 6D.

Thus, microwell arrays containing microcontact-printed Protein A is a versatile tethering system in that it can be incubated with any Fc-chimeric to give microwells with a properly oriented (i.e., surface exposed and available for interaction with a cell), immobilized protein localized to the bottom of each microwell.

### Example 9

### HSC Division Kinetics Change in Response to Selected Soluble and Immobilized Protein Cues

The effects of selected soluble and immobilized proteins on the proliferation kinetics of LKS-CD150+ (designated hereon as HSCs) were systematically tested. To maximize the sensitivity in detecting responses to individual factors, basal growth factor conditions supplemented with a series of seven different soluble protein morphogens or cytokines, or six Fc-chimeric transmembrane proteins were tested. All factors were tested separately, but simultaneously, in multiple experiments in 96-well plates such as shown in FIGS. 8A-8C.

The addition of single proteins to the basal medium markedly altered proliferation kinetics, as is evident from the distribution of total cells per microwell over a period of 4 days in culture, as shown in FIG. 8A.

The kinetic proliferation profiles, quantified by the distribution of hematopoietic stem cell progeny generated per microwell per day in response to specific proteins, revealed four distinct patterns, as shown in FIG. 8A. Most proteins (IGF-2, FGF-1, Angl, I-CAM, VE-Cad, P-Sel, V-CAM) exhibited a proliferation profile similar to that of the basal media, i.e., they had no noticeable effect (Type I). By contrast, one protein, Wnt3a, resulted in relatively small clone sizes of primarily one or two cells per microwell (Type II). Two proteins (TPO, IL11) resulted in relatively large clone sizes of >8 cells/microwell (Type II). In general, Types I-III exhibited a prevalence of clones with even numbers of 2, 4 and 8 cells. However, for three proteins (Shh, Jag-1, and N-cad), designated as Type IV, the number of clones with an odd number of 3 cells per microwell was increased above basal Type I conditions, indicative of a higher frequency of asynchronous division of daughter cells.

For Type IV proteins, an additional analysis was performed to determine the percentage of microwells that contained 3 cells at 24-hour time intervals and an increase in the proportion of microwells with three cells was consistently observed over a period of one week compared to basal conditions, as shown in FIG. 8C. Care was taken to only score novel appearances of 3 cells per microwell at each time point to avoid counting the same data twice. A histogram for proteins of Types I-IV is shown in FIG. 8B depicting the relative proportions of microwells having non-dividing cells (1 cell), slow dividing clones (2 cells), fast dividing clones (>4 cells) or asynchronously dividing clones (3 cells). From these data, the four distinct proliferative patterns are apparent. Representative proteins of the three types that differed from basal were selected for further analysis, namely Wnt3a (Type II), TPO (Type II), and N-cadherin (Type IV), of which the first two were soluble, and the last was tethered.

An analysis of the detailed time course, in particular the time between divisions in culture, revealed profound differences among the three types of proteins. Time-lapse experiments were performed with one-hour time intervals for a period of up to 7 days. Compared to basal conditions, TPO-exposed HSCs exhibited a relatively homogeneous distribution, entering their first division on average at 37 hrs. Most cells that underwent a first division in the presence of TPO divided a second time, with an average time to division of 21 hours. Notably, TPO proliferation kinetics (FIG. 8B) resembled those observed with multipotent progenitors with similar peaks and times to first division and between first and second divisions.

In contrast, cells exposed to Wnt3a and N-cadherin revealed a higher degree of heterogeneity, with some cells dividing almost immediately, and others entering their first division after as much as 80 hr.

Notably, HSCs exposed to Wnt3a and N-cadherin displayed average times between first and second divisions and time to first division. (50 hr versus 47 hr, and 47 hr versus 34 hr, respectively) that were not reduced, but instead somewhat prolonged. These results show that exposure of single HSCs to single extrinsic cues had a marked effect on stem cell proliferation kinetics in vitro.

### Example 10

### Slow Cell Proliferation Kinetics Induced by Wnt3a Correlate with Long-Term Reconstitution In Vivo

The disparate proliferation behaviors observed with TPO, Wnt3a and N-cadherin herein indicated that HSCs cultured in the presence of these three factors might have different biological properties. Accordingly, cells exposed to these factors were tested with respect to self-renewal, and multipotency, and engraftment was assessed by long-term blood reconstitution. 100 HSCs were seeded in microwell arrays, exposed to TPO, Wnt3a, N-cad, or basal medium alone in culture for more than 4 days, and all progeny were harvested, pooled, and transplanted into lethally irradiated hosts, as schematically shown in FIG. 9A.

After 6 months, a high efficiency of reconstitution with robust peripheral blood chimerism was obtained in mice transplanted with GFP⁺HSCs that had been cultured in the presence of Wnt3a (6/9, with up to 93% PB chimerism), or N-cadherin (4/5, with up to 95% PB chimerism), whereas a low efficiency of reconstitution with low chimerism was obtained for the basal medium control (1/9, up to 5% PB chimerism) and TPO (2/9, up to 21% PB chimerism), as shown in FIG. 9B. In addition, donor-derived peripheral blood chimerism persisted for six months in all mice reconstituted with cells exposed to Wnt3a or N-cad, but declined progressively in mice reconstituted with cells exposed to basal medium or TPO (FIG. 9B). Wnt3a- or N-cad-treated cells yielded normal lymphoid and myeloid ratios, whereas cells exposed to basal medium and TPO-treated cells, gave rise primarily to lymphoid lineages.

These differences were even more pronounced upon secondary transplantation of HSCs from reconstituted mice into lethally irradiated recipients as shown in FIG. 9D. Whereas uncultured Wnt3a- and N-cad-treated cells led to reconstitution in most secondary recipients (17/17, up to 95% PB chimerism; 20/21, up to 91% PB chimerism; 3/4 up to 67% PB chimerism, respectively), none (0/3) of the TPO-treated cells from the poorly reconstituted primary transplants led to successful reconstitution upon secondary transplantation, and cells exposed to the basal medium yielded such low reconstitution that secondary transplants were not possible.

These data show that exposure to Wnt3a or N-cadherin in vitro in hydrogel microwells leads to retention of stem cell function. These data also provide evidence that both the rate and synchrony of stem cell division induced by single extrinsic factors in vitro correlated with in vivo HSC reconstitution potential in mice, indicating that these characteristics could serve as predictors of maintenance of stem cell function.

### Example 11

### Evidence for Self-Renewal: Wnt3a and N-Cadherin Maintain Stem Cell Multipotency After Division in Culture

The effect of Wnt3a or N-cadherin shown in FIGS. 8A-8D could result either from a retention of stem cell function in non-dividing cells or from self-renewal and the production of another stem cell in the course of cell division in culture. To distinguish between these two possibilities, the in vivo function of HSCs that never divided (singlets), divided once (doublets) or divided more than three times (clones) was analyzed.

For this purpose, a series of transplantation experiments was carried out using micromanipulation to harvest HSC progeny from individual microwells after exposure to TPO, Wnt3a or N-cadherin, as schematically shown in FIG. 1A. Notably, TPO-treated singlets could not be tested, as cells that did not undergo division within 4 days were exceedingly rare, except when they formed giant megakaryocytes. Strikingly, upon transplantation of singlets exposed to N-cadherin (10 cells transplanted per lethally irradiated mouse), long-term blood reconstitution was detected in 1 of 4 mice. These results demonstrated that stem cell multipotency can be maintained for up to one week in the absence of cell division in culture in the presence of N-cadherin, but not Wnt3a.

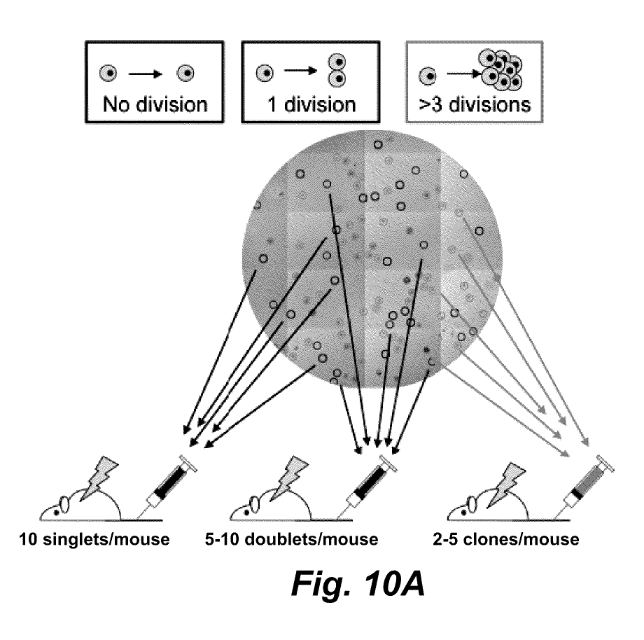

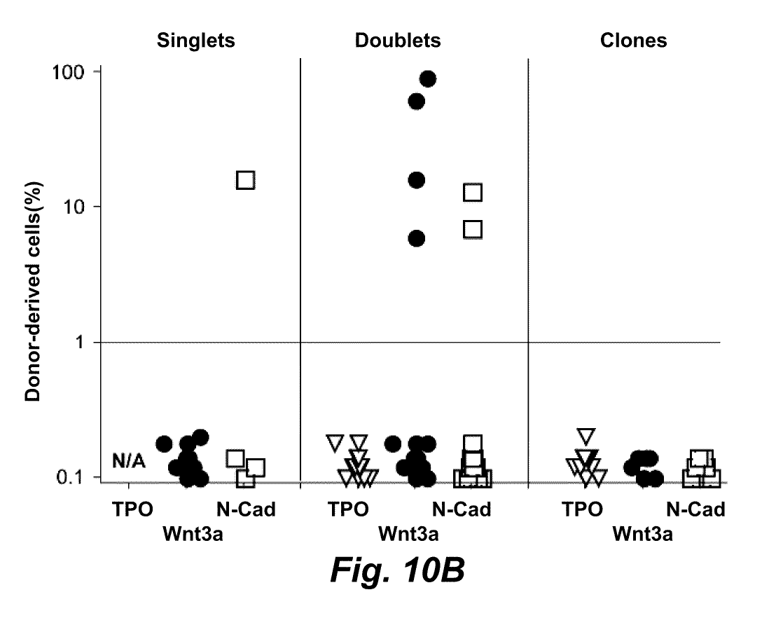

This experiment required monitoring the microwell cultures by continuous time-lapse microscopy to ascertain when division occurred. Notably, none of the animals (0/16) transplanted with a total of 100 TPO-stimulated doublets exhibited blood reconstitution, as shown in FIG. 10B. In contrast, out of the Wnt3a-stimulated HSC doublets transplanted into 19 recipient animals, 3 mice exhibited high reconstitution potential with up to 92% peripheral blood chimerism (115 doublets transplanted; 3 of 19 transplanted mice), comparable to N-cadherin stimulated cells (90 doublets transplanted; 2 of 15 transplanted mice).

These experiments demonstrated that soluble Wnt3a or immobilized N-cadherin can maintain stem cells by self-renewal, whereas TPO cannot. By contrast, transplantation of larger clone sizes (those that had undergone multiple rounds of replication) never resulted in bone marrow reconstitution, irrespective of the factors to which HSCs were exposed, suggesting that even in the presence of Wnt3a or N-cadherin, such cells characterized by a faster proliferation rate in culture had lost their stem cell capacity (i.e. the ability to regenerate the hematopoietic system). These results show that stem cell function is maintained upon one division in the presence of Wnt3a or N-cadherin, and provide evidence of self-renewal in response to single proteins in vitro.

### Example 12

### Retrospective Fate Analysis Via Multiplex Single Cell Nested PCR

To determine whether the Wnt3a- and TPO-induced differences in HSC proliferation correlated with changes in gene expression, cultured cells were compared with freshly isolated uncultured cells using multiplex, single-cell nested RT-PCR. Tie-2 and Gata 3 were selected as they are co-expressed in 78% of uncultured LKS-CD150+, but not uncultured LKS-CD150-cells, and are therefore more characteristic of stem cells.

Of Wnt3a-stimulated cells, 66% retained the expression profile of uncultured stem cells, in contrast to only 22% and 0% of cells exposed to basal medium and TPO, respectively. These results show that after 7 days, Wnt3a-treated cells approach uncultured cells in maintaining this stem cell gene expression profile.

### Single Cell Collection.

Single cells were directly sorted via FACS into PCR tubes containing 9 μl aliquots of RT-PCR lysis buffer. The buffer components included commercial RT-PCR buffer (SuperScript One-Step RT-PCR Kit Reaction Buffer, Invitrogen), RNase inhibitor (Protector RNase Inhibitor, Roche) and 0.15% IGEPAL detergent (Sigma). After a short pulse-spin, the PCR-tubes were immediately shock-frozen and stored at −80° C. for subsequent analysis.

### Two-Step Multiplex Single Cell RT-PCR.

Cell lysates were first reverse-transcribed using three pairs of gene-specific primers as described by the manufacturer (SuperScript One-Step RT-PCR Kit, Invitrogen). Briefly, the RT-PCR was performed in the same PCR cell-lysis tubes by addition of a RT-PCR-reaction mix containing the gene-specific primer pairs and RNase inhibitor. Genomic products were excluded by designing and using intron-spanning primer sets for the first and second round PCR (see Table 2). The expected PCR-product sizes for the first and second round were around 450 bp (external primers) and 250 bp (internal primers), respectively. The reverse transcription reactions were done at 55° C. for 30 min, and followed by a 2-min step at 94° C. Subsequently, 30 cycles of PCR amplification were performed as follows: 94° C. for 20 sec; 55° C. for 25 sec; 68° C. for 30 sec. In the final PCR step, the reactions were incubated for 3 min at 68° C. The completed reactions were stored at 4° C.

In a second step, the completed RT-PCR reaction from the first step was diluted 1:1 with water. One percent of these reactions were replica transferred into new reaction tubes for the second round of PCR, which was performed for each of the three genes separately using fully nested gene-specific internal-primers as indicated by the manufacturer in a total reaction volume of 20 μl (Platinum Taq Super-Mix HF, Invitrogen). Thirty cycles of PCR amplification were performed as follows: 94° C. for 20 sec; 58.5° C. for 20 sec; 68° C. for 20 sec. In the final PCR step, the reactions were incubated for 3 min at 68° C. The completed reactions were stored at 4° C. Finally, the second round PCR products were subjected to gel electrophoresis using one fifth of the reaction volumes and 1.4% agarose gels.

TABLE 2
Primer sequences utilized for single cell PCR

| Multi- | Nested Primer Sets | |
|---|---|---|
| plex | External Primers | Internal Primers |
| genes | [5′-3′] | [5′-3′] |
| HPRT | GCTCGAGATGTCATGAAGGAG | GTTCTTTGCTGACCTGCTGG |
| | (SEQ ID NO.: 1) | (SEQ ID NO.: 2) |
| | TCCAACACTTCGAGAGGTCC | GGCTGTACTGCTTAACCAGG |
| | (SEQ ID NO.: 3) | (SEQ ID NO.: 4) |
| GATA-3 | GAAGCTCAGTATCCGCTGAC | CATCGATGGTCAAGGCAACC |
| | (SEQ ID NO.: 5) | (SEQ ID NO.: 6) |
| | GGGAGGGTGAAGAGATGAGG | GCCAGAGAAGAGGATGAAGC |
| | (SEQ ID NO.: 7) | (SEQ ID NO.: 8) |
| Tie-2 | GAAACATCCCTCACCTGCAT | ATGAACCAGCACCAAGATCC |
| | (SEQ ID NO.: 9) | (SEQ ID NO.: 10) |
| | TGCGGCAAGTGAACTTCTAA | CCCTGTCCACGGTCATAGTT |
| | (SEQ ID NO.: 11) | (SEQ ID NO.: 12) |

We claim:

1. A system for isolating or culturing a cell, the system comprising a hydrogel film comprising a cross-linked polymeric composition has the characteristic of hydrating to form a hydrogel and having a topographical feature or a plurality of topographical features, wherein each topographical feature has a surface capable of receiving and immobilizing at least one biomolecule species thereon.

2. The system of claim 1, wherein the hydrogel film is hydrated as a hydrogel.

3. The system of claim 1, wherein a biomolecule species is immobilized to the cross-linked polymeric composition.

4. The system of claim 3, wherein the at least one biomolecule species is selected from the group consisting of: a polypeptide, a peptide, an oligonucleotide, and a small molecule.

5. The system of claim 3, wherein the biomolecule species is selected from the group consisting of: Wnt3a, N-cadherin, thrombopoietin, erythropoietin, granulocyte-macrophage colony stimulating factor, granulocyte colony stimulating factor, macrophage colony stimulating factor, thrombopoietin, stem cell factor, interleukin-1, interleukin-2, interleukin-3, interleukin-6, interleukin-7, interleukin-15, Flt3L, leukemia inhibitory factor, insulin-like growth factor, insulin, and recombinant insulin.

6. The system of claim 1, wherein the cross-linked polymeric composition is selected from the group consisting of: a poly(ethylene glycol), a polyaliphatic polyurethane, a polyether polyurethane, a polyester polyurethane, a polyethylene copolymer, a polyamide, a polyvinyl alcohol, a polypropylene glycol, a polytetramethylene oxide, a polyvinyl pyrrolidone, a polyacrylamide, a poly(hydroxyethyl acrylate), and a poly(hydroxyethyl methacrylate).

7. The system of claim 1, wherein the cross-linked polymeric composition is formed from at least two precursor compounds in a ratio whereby, when the precursors are cross-linked to form the polymeric composition, a surface of a topographical feature adapted to immobilize a polypeptide or a tether thereto.

8. The system of claim 1, wherein the cross-linked polymeric composition is synthesized from at least two precursor compounds wherein one precursor compound comprises n nucleophilic groups, and a second precursor compound comprises m electrophilic groups, wherein n and m are each at least 2 and the sum (n+m) is at least five.

9. The system of claim 8, wherein the cross-linked polymeric composition is synthesized from at least two precursor components using a Michael-type addition reaction.

10. The system of claim 8, wherein the n nucleophilic groups are thiol groups.

11. The system of claim 8, wherein the m electrophilic groups are conjugated unsaturated groups.

12. The system of claim 1, wherein the plurality of topographical features is an array of microwells.

13. The system of claim 1, wherein a surface of a topographical feature comprises a reactive functional group of the cross-linked polymeric composition, wherein the reactive functional group is capable of binding to a biomolecule species desired to be immobilized on the surface of the topographical feature.

14. The system of claim 13, wherein the reactive functional group is selected from the group consisting of: a succinimidyl active ester, an aldehyde, a thiol, and a thiol-selective group.

15. The system of claim 1, wherein a topographical feature has a tether immobilized thereon, wherein the tether is adapted to selectively bind to the biomolecule species desired to be immobilized on the surface of the topographical feature.

16. The system of claim 15, wherein the tether is selected from the group consisting of: a peptide, a polypeptide, and a non-peptide linker.

17. The system of claim 15, wherein the tether is selected from the group consisting of: a heterofunctional PEG, Protein A, Protein G, an immunoglobulin, streptavidin, neutravidin, biotin, a linker capable of forming a complex with a metal ion, and a transglutaminase substrate.

18. The system of claim 15, wherein the tether is Protein A or Protein G, and the biomolecule species bound thereto is a polypeptide comprising an immunoglobulin Fc region and a region capable of interacting with a cell disposed in the topographical feature.

19. The system of claim 1, wherein the hydrogel film is disposed in a well of a multi-well tissue culture plate.

20. A microcontact printing method of preparing a hydrogel, comprising:
(a) providing a template comprising a negative topographical feature or a plurality of negative topographical features, wherein each negative topographical feature defines a topographical feature desired to be formed in a hydrogel, and wherein the negative topographical feature or features has on a surface thereof a biomolecule species desired to be transferred to a hydrogel, a tether capable of selectively binding to said biomolecule species, or a combination thereof;
(b) delivering to the template a hydrogel polymer precursor composition;
(c) polymerizing the hydrogel polymer precursor composition to form a hydrogel film; and
(d) removing the hydrogel film from the template, thereby transferring the biomolecule species, the tether, or a combination thereof, to a surface of the topographical feature molded in the hydrogel film.

21. The method of claim 20, wherein each topographical feature molded in the hydrogel is a microwell.

22. The method of claim 20, further comprising: hydrating the hydrogel film, thereby forming a hydrogel.

23. The method of claim 20, wherein the biomolecule species is selected from the group consisting of: a polypeptide, a peptide, an oligonucleotide, and a small molecule.

24. The method of claim 23, wherein the biomolecule species is selected from the group consisting of: Wnt3a, N-cadherin, thrombopoietin, erythropoietin, granulocyte-macrophage colony stimulating factor, granulocyte colony stimulating factor, macrophage colony stimulating factor, thrombopoietin, stem cell factor, interleukin-1, interleukin-2, interleukin-3, interleukin-6, interleukin-7, interleukin-15, Flt3L, leukemia inhibitory factor, insulin-like growth factor, insulin, and recombinant insulin.

25. The method of claim 20, wherein, if a tether is transferred from the template to a surface of a topographical feature, the method further comprises delivering to the topographical feature of the hydrogel a composition, said composition comprising a biomolecule species desired to be immobilized on the surface of the microwells, thereby selectively binding the biomolecule species to the tether and immobilizing the biomolecule species to the surface of the topographical feature.

26. The method of claim 20, wherein a surface of a topographical feature or of a plurality of topographical features comprises a reactive functional group of the cross-linked polymeric composition, wherein the reactive functional group is capable of binding to the biomolecule species desired to be immobilized on the topographical feature or plurality of topographical features.

27. The method of claim 26, wherein the reactive functional group is selected from the group consisting of: a succinimidyl active ester, an aldehyde, a thiol and a thiol-selective group.

28. The method of claim 20, wherein a surface of a topographical features has a tether immobilized thereon, wherein the tether is capable of selectively binding to the biomolecule species desired to be immobilized on the surface of the topographical feature.

29. The method of claim 28, wherein the tether is selected from the group consisting of: a peptide, a polypeptide, and a non-peptide linker.

30. The method of claim 28 wherein the tether is selected from the group consisting of: a heterofunctional PEG, Protein A, Protein G, an immunoglobulin, streptavidin, neutravidin, biotin, a linker capable of forming a complex with a metal ion, and a transglutaminase substrate.

31. The method of claim 28, wherein the tether is Protein A or Protein G, and the biomolecule species bound thereto comprises an immunoglobulin Fc region and a region capable of interacting with a cell disposed in the microwell of the topographical feature.

32. The method of claim 20, wherein the hydrogel polymer precursor composition comprises at least two precursor compounds in a ratio whereby when the precursors are cross-linked to form the polymer the surface of the microwells of the microwell array is capable of immobilizing a polypeptide or a tether thereto.

33. The method of claim 20, wherein the hydrogel film is disposed in a well of a multi-well tissue culture plate.

34. A method of isolating individual cells from a population of cells, comprising:
(a) providing a hydrogel system disposed in a well of a multi-well tissue culture plate, wherein the hydrogel comprises a hydrated cross-linked polymer having an array of topographical features indented therein, and wherein a surface of each of the topographical features has at least one biomolecule species immobilized thereon;
(b) delivering a cell suspension of isolated cells to the well of the multi-well tissue culture plate, whereby the cells of the suspension descend under gravity into the multiplicity of microwells, and wherein the cell density of the cell suspension is adjusted whereby at least one well of the multiplicity of wells receives a single cell; and
(c) incubating the hydrogel under conditions favorable for proliferation of the cells.

35. The method of claim 34, wherein the topographical feature is a microwell.

36. The method of claim 34, wherein the cell suspension comprises a population of stem cells.

37. The method of claim 36, wherein the stem cells of the population of stem cells is selected from the group consisting of: a hematopoietic stem cell, a hematopoietic progenitor cell, an adult stem cell, an embryonic stem cell, and a cancer stem cell.

38. The method of claim 34, wherein a biomolecule species is immobilized by a tether to a surface of a topographical feature.

39. The method of claim 34, wherein the at least one biomolecule species is selected from the group consisting of: a polypeptide, a peptide, an oligonucleotide, and a small molecule.

40. The method of claim 39, wherein the biomolecule species is selected from the group consisting of: Wnt3a, N-cadherin, thrombopoietin, erythropoietin, granulocyte-macrophage colony stimulating factor, granulocyte colony stimulating factor, macrophage colony stimulating factor, thrombopoietin, stem cell factor, interleukin-1, interleukin-2, interleukin-3, interleukin-6, interleukin-7, interleukin-15, Flt3L, leukemia inhibitory factor, insulin-like growth factor, insulin, and recombinant insulin.

41. The method of claim 40, wherein a surface of a topographical feature comprises a reactive functional group of the cross-linked polymeric composition, wherein the reactive functional group is capable of binding to the biomolecule species desired to be immobilized on the surface of the topographical feature.

42. The method of claim 40, wherein the reactive functional group is selected from the group consisting of: a succinimidyl active ester, an aldehyde, a thiol and a thiol-selective group.

43. The method of claim 38, wherein the tether is selected from the group consisting of: a peptide, a polypeptide, and a non-peptide linker.

44. The method of claim 38, wherein the tether is selected from the group consisting of: a heterofunctional PEG, Protein A, Protein G, an immunoglobulin, streptavidin, neutravidin, biotin, a linker capable of forming a complex with a metal ion, and a transglutaminase substrate.

45. The method of claim 38, wherein the tether is Protein A or Protein G, and the biomolecule species bound thereto comprises an immunoglobulin Fc region and a region capable of interacting with a cell disposed in the microwell of the topographical feature.

46. A method for determining the proliferative outcome of transplanting a stem cell into a recipient host, comprising;
(a) delivering a population of cells to a plurality of microwells indented in a hydrogel, wherein an interior surface of each microwell of the plurality of microwells has a biomolecule species immobilized thereon, and wherein at least some of the microwells of the multiplicity of microwells receive a single cell from the population of cells;
(b) monitoring the proliferation of the isolated single cells by time-lapse photography;
(c) correlating the proliferation of the cells to the proliferative outcome of a stem cell transplanted into a recipient host; and
(d) identifying those cells in a microwell having the characteristic of regenerating a hematopoietic system when transplanted into a recipient host.

47. The method of claim 46, wherein the stem cell is a hematopoietic stem cell.
